(19) United States
(12) Patent Application Publication
(10) Pub. No.: US 2010/0226983 A1
(43) Pub. Date: Sep. 9, 2010

(54) Compositions for treatment and prevention of acne, methods of making the compositions, and methods of use thereof

(75) Inventors: Joyce A. Sutcliffe, West Newton, MA (US); Susan M. Ciotti, Ann Arbor, MI (US); James R. Baker, JR., Ann Arbor, MI (US)

Correspondence Address: FOLEY AND LARDNER LLP;SUITE 500, 3000 K STREET NW, WASHINGTON, DC, 20007, US

(73) Assignee: NanoBio Corporation

(21) Appl. No.: 12/656,421

(22) Filed: Jan. 28, 2010

Related U.S. Application Data
(60) Provisional application No. 61/147,960, filed on Jan. 28, 2009.

(51) Int. Cl. A61K 9/107; A61K 31/235; A61K 31/60; A61K 31/045

(52) U.S. Cl. 424/484; 514/544; 514/159; 514/724

(57) ABSTRACT

The present invention relates to methods for treating and preventing acne or P. acnes infection in a subject comprising topically administering to the subject in need thereof an anti-acne nanoemulsion composition.

### CROSS-REFERENCE TO RELATED APPLICATION

This application claims priority from U.S. Provisional Patent Application No. 61/147,960, filed Jan. 28, 2009. The contents of that application is incorporated herein by reference in its entirety.

### FIELD OF THE INVENTION

The present disclosure relates to compositions and methods for preventing, and/or treating acne or killing, and/or inhibiting the growth of Propionibacterium acnes. The method comprises topically administering to a subject in need thereof a nanoemulsion composition having anti-acne properties.

### BACKGROUND OF THE INVENTION

### A. Acne and P. acnes Infection

Acne is a chronic inflammatory disease affecting more than 85% of teenagers, and continuing into adulthood in some populations. Some individuals suffer from acne into their thirties, forties and beyond. Acne is most frequently found on the face and upper neck, but also found on the chest, back, shoulders and upper arms. Acne lesions can develop into comedo, papule, pustule, lupus, nodule, or scars.

Acne is a disease of pilosebaceous units in the skin. Although the cause of acne is not fully understood, some factors have been linked to acne, such as genetic history, hormone level, skin inflammation, etc. In acne, excessive sebum production occurs in the sebaceous gland. This causes hyperkeratinization of the hair follicle and prevents normal shedding of the follicular keratinocytes. This results in obstruction of the hair follicle and subsequent accumulation of lipids and cellular debris in the blocked hair follicle. Colonization of an anaerobic gram-positive bacterium, Propionibacterium species., e.g., Propionibacterium acnes, occurs in the blocked follicle. This bacteria is present on most human skin and lives on fatty acids in the pilosebaceous unit. Infection of the hair follicle results in inflammation. Inflammation is further enhanced by rupture of the hair follicle and release of lipids, bacteria, and fatty acids into the dermis.

### B. Conventional Treatment for Acne

Conventional treatment for acne includes topical or oral administration of bactericidals, benzoyl peroxide, triclosan bekeratolytics, e.g., salicylic acid, and chlorhexidine, acitretin, alcloxa, aldioxa, allantoin, dibenzothiophene, etarotent, etretinate, motretinide, nordihydroguaiaretic acid, podofilox, podophyllum resin, resorcinalm resorcinol monoacetate, sumarotene, tetroquinone, retinoids, e.g., tretinoin, isotretinoin, adapalene and tazarotene, antibiotics, e.g., erythromycin, clindamycin, tetracycline, minocycline, doxycycline, hormones, e.g., estrogen, and progesterone, and combination products, e.g., stievamycin, Murad®, Benzaclin® and Benzamycin®. Other anti-acne ingredients include Ascorbyl Tetraisopalmitate, Dipotassium Glycyrrhizinate, Ascorbyl Tetraisopalmitate, Niacinamide, alpha bisabolol. All of these ingredients have properties that help to reduce and control acne, and acne related problems such as sebum production. Herbal medicines are also used to treat acne and include Tea Tree Oil red clover, lavender, leaves of strawberry, chaste tree berry extract, burdock root, dandelion leaves, milk thistle, papaya enzymes, burdock and dandelion, eucalyptus, thyme, witch hazel, sage oil, camphor, cineole, rosmarinic acid and tannins in the sage oil. These various treatments for acne may have only temporary effects, and may cause drug-resistance or other undesirable side effects, such as allergy, skin redness, or skin hypersensitivity.

Orally administered drugs are generally more effective than topically applied drugs, but because they act systemically rather than locally, the side effects of orally administered drugs can limit their use.

### C. Background Regarding Nanoemulsions

Prior teachings related to nanoemulsions are described in U.S. Pat. No. 6,015,832, which is directed to methods of inactivating a Gram-positive bacteria, a bacterial spore, or a Gram-negative bacteria. The methods comprise contacting the Gram-positive bacteria, bacterial spore, or Gram-negative bacteria with a bacteria-inactivating (or bacterial-spore inactivating) emulsion. U.S. Pat. No. 6,506,803 is directed to methods of killing or neutralizing microbial agents (e.g., bacteria, virus, spores, fungus, on or in humans using an emulsion. U.S. Pat. No. 6,559,189 is directed to methods for decontaminating a sample (human, animal, food, medical device, etc.) comprising contacting the sample with a nanoemulsion. The nanoemulsion, when contacted with bacterial, virus, fungi, protozoa, or spores, kills or disables the pathogens. The antimicrobial nanoemulsion comprises an oil, quaternary ammonium compound, one of ethanol/glycerol/PEG, a surfactant, and water. U.S. Pat. No. 6,635,676 is directed to two different compositions and methods of decontaminating samples by treating a sample with either of the compositions. Composition 1 comprises an emulsion that is antimicrobial against bacteria, virus, fungi, protozoa, and/or spores. The emulsions comprise an oil and a quaternary ammonium compound. U.S. Pat. No. 7,314,624 is directed to methods of inducing an immune response to an immunogen comprising treating a subject via a mucosal surface with a combination of an immunogen and a nanoemulsion. The nanoemulsion comprises oil, ethanol, a surfactant, a quaternary ammonium compound, and distilled water. US-2005-0208083-A1 and US-2006-0251684-A1 are directed to nanoemulsions having droplets with preferred sizes. US-2007-0054834-A1 is directed to compositions comprising quaternary ammonium halides and methods of using the same to treat infectious conditions. The quaternary ammonium compound may be provided as part of an emulsion. Finally, US-2007-0036831-A1 is directed to nanoemulsions comprising an anti-inflammatory agent.

There is a need in the art for improved treatment options for patients affected by acne. Specifically, there is a need in the art for an effective topical agent to treat and prevent acne and/or infection by P. acnes. The present invention satisfies these needs.

### SUMMARY OF THE INVENTION

The present invention provides methods and compositions for treating and/or preventing acne and/or infection by P. acnes in a subject comprising administering a nanoemulsion topically to the subject. The nanoemulsion comprises droplets having an average diameter of less than about 3 microns, and the nanoemulsion droplets comprise an aqueous phase, at least one oil, at least one surfactant, and at least one organic solvent.

Surprisingly, it was discovered that the topically applied nanoemulsions have potent cidal activity against P. acnes and synergy with other agents commonly used to treat acne. The composition of the invention allows for targeted delivery into the pilosebaceous unit, the site of acne pathogenesis. This is significant, as a topically applied, and therefore local, site-specific activity, is highly preferable over an orally administered, and therefore systemic activity. Moreover, the nanoemulsions are able to enhance delivery, and thus effectiveness, of other topical anti-acne agents incorporated into the nanoemulsion, thereby enhancing the efficacy and reducing the detrimental side effects of the other anti-acne agents.

In certain embodiments of the invention, the nanoemulsion can have an increased viscosity to aid in permeation of the nanoemulsion into the dermis and epidermis.

In other embodiments of the invention, the nanoemulsion at the time of topical application is at room temperature or warmer.

The nanoemulsion comprises droplets having an average particle size of less than about 3 microns, and the nanoemulsion comprises water, at least one oil, at least one surfactant, and at least one organic solvent. In one embodiment of the invention, the surfactant present in the nanoemulsion is a cationic surfactant. In another embodiment of the invention, the nanoemulsion further comprises a chelating agent. In one embodiment of the invention, nanoemulsions from the present invention, or those derived from the nanoemulsions of the present invention, are diluted. The diluted samples can then be tested to determine if they maintain the desired functionality, such as surfactant concentration, stability, particle size, and/or anti-infectious activity (e.g., antimicrobial activity against P. acnes).

In some embodiments, a second anti-acne agent is incorporated into the nanoemulsion to achieve improved delivery, efficacy and or tolerability of the second anti-acne agent. Preferably, the second anti-acne agent is selected from the group consisting of benzoyl peroxide, salicylic acid, acitretin, alcloxa, aldioxa, allantoin, dibenzothiophene, etarotent, etretinate, motretinide, nordihydroguaiaretic acid, podofilox, podophyllum resin, resorcinalm resorcinol monoacetate, sumarotene, tetroquinone, triclosan, chlorhexidine, azelaic acid, hydrocortisone, sodium hyaluronate, sulfur, urea, retinoids or retinoid derivatives, e.g., tretinoin, isotretinoin, antibiotics, e.g., erythromycin, clindamycin, tetracycline, minocycline, doxycycline, meclocycline, hormones, e.g., estrogen, and progesterone, adapalene and tazarotene and combination products, e.g., stievamycin, Murad®, Benzaclin® and Benzamycin®, and any combination thereof. Other anti-acne ingredients include Ascorbyl Tetraisopalmitate, Dipotassium Glycyrrhizinate, Ascorbyl Tetraisopalmitate, Niacinamide, and alpha bisabolol. All of these skin care ingredients have properties that help to reduce and control acne, and acne-related problems such as sebum production. Herbal medicines are also used to treat acne and include Tea Tree Oil red clover, lavender, leaves of strawberry, chaste tree berry extract, burdock root, dandelion leaves, milk thistle, papaya enzymes, burdock and dandelion, eucalyptus, thyme, witch hazel, sage oil, camphor, cineole, rosmarinic acid and tannins in the sage oil.

Inclusion of a second antibiotic into the nanoemulsion should reduce the potential for resistance development towards either the nanoemulsion or added antibiotic. The nanoemulsion may further comprise anti-comdeogenic, anti-inflammatory, keratolytics, sebum supressors as disclosed in PCT publication No. WO/01/56556 A2. One skilled in the art will understand that any suitable or desirable second active agent useful in treating acne can be incorporated into the nanoemulsion of this invention.

Preferably, the nanoemulsions for topical administration are in the form of any pharmaceutically acceptable dosage form, including but not limited to, ointments, creams, emulsions, lotions, gels, liquids, bioadhesive gels, sprays, shampoos, aerosols, pastes, foams, sunscreens, capsules, microcapsules, or in the form of an article or carrier, such as a bandage, insert, syringe-like applicator, pessary, powder, talc or other solid, shampoo, cleanser (leave on and wash off product), and agents that favor penetration within pilosebaceous unit, the epidermis, the dermis and keratin layers. The nanoemulsion is capable of effectively treating, preventing, and/or curing acne, without being systemically absorbed and without significantly irritating the skin.

The foregoing general description and following brief description of the drawings and the detailed description are exemplary and explanatory and are intended to provide further explanation of the invention as claimed. Other objects, advantages, and novel features will be readily apparent to those skilled in the art from the following detailed description of the invention.

### BRIEF DESCRIPTION OF THE DRAWINGS

FIG. 1 illustrates the cross-section view of the pilosebaceous unit in human cadaver skin and hamster ear after application of nanoemulsion plus fluorescein

FIG. 2 shows in vitro skin permeation of nanoemulsion formulations into the epidermal layer of pig abdominal skin at 24 hours after a single topical application of 100 μl/cm².

FIG. 3 shows in vitro permeation of nanoemulsion formulations in pig abdominal skin at 12 and 24 hours after a single topical application of 100 μl/cm².

FIG. 4 shows the in vitro MBC of a nanoemulsion (NB-003) with and without (+/−) the presence of 25% sebum. The figure shows that the MBC of the nanoemulsion rises 500-fold in the presence of sebum, unless additional EDTA is added to the formulation.

FIG. 5 shows the effect the concentration of a nanoemulsion has on the particle size and viscosity of the nanoemulsion. With a decrease in concentration of the active, viscosity (cP) declines (triangles), whereas the particle size remains constant (bars).

FIG. 6 shows the results of a permeation study utilizing pig skin epidermis with 5 skin sections (n=5) following administration of a nanoemulsion (NB-003) twice daily (BID). Higher viscosity (greater than 1000 cps) nanoemulsions (e.g., 0.8% NB-003) were found to enhance permeation of the nanoemulsion into the epidermis.

FIG. 7 shows the results of a permeation study utilizing pig skin dermis with 5 skin sections (n=5) following administration of a nanoemulsion (NB-003) twice daily (BID). Higher viscosity (greater than 1000 cps) nanoemulsions (e.g., 0.8% NB-003) were found to deliver three times the amount of the surfactant, cetylpyridinium chloride (CPC) to the dermis as compared to a lower viscosity nanoemulsion (e.g., 0.25% NB-003).

FIG. 8 shows the effect of storage temperature of a nanoemulsion (e.g., NB-003) on the in vitro activity of the nanoemulsion against P. acnes in the presence of sebum.

### DETAILED DESCRIPTION OF THE PREFERRED EMBODIMENTS

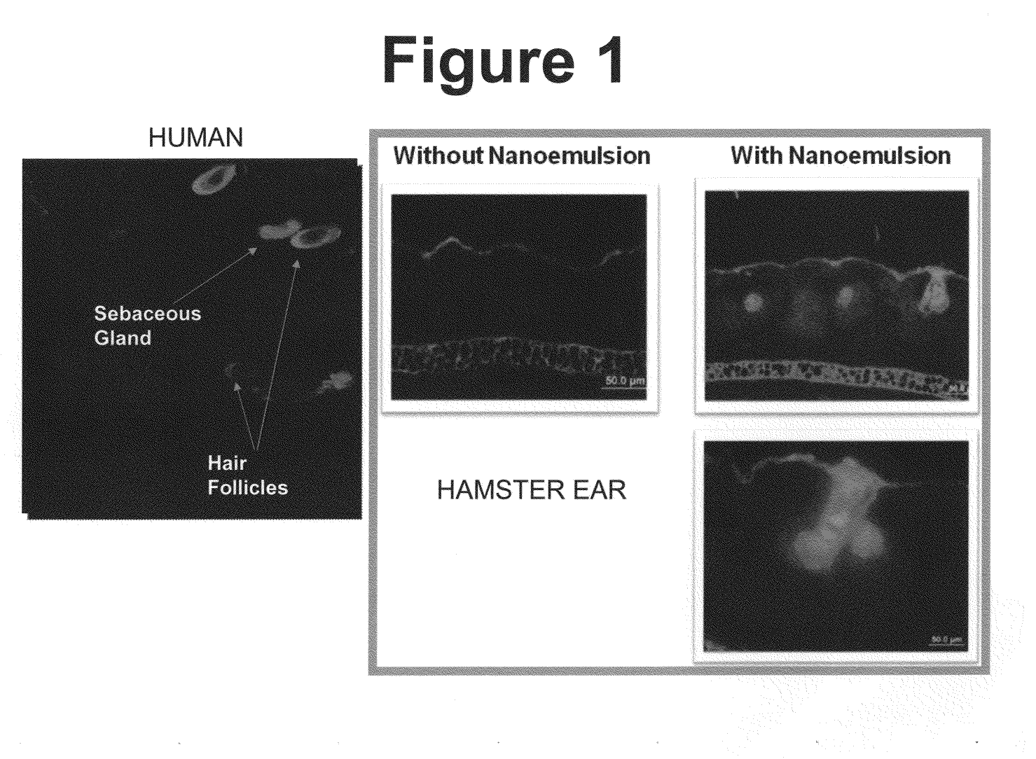

The present disclosure provides methods and compositions for treating, preventing, and/or curing acne and/or infection by P. acnes in a subject comprising administering topically or to the subject a nanoemulsion. The nanoemulsion comprises droplets having an average diameter of less than about 3 microns, and the nanoemulsion droplets comprise an aqueous phase, at least one oil, at least one surfactant, and at least one organic solvent. The delivery of nanoemulsions is targeted to the site of acne pathogenesis. i.e., the pilosebaceous unit. See FIG. 1.

Propionibacterium acnes, a gram-positive, non-spore forming, anaerobic bacillus, is one of the primary factors involved in the pathogenesis of acne vulgaris. It is the predominant microorganism of the pilosebaceous glands of human skin, with up to 10 million viable organisms isolated from a single sebaceous unit. Although aerotolerant, P. acnes typically grows in the anaerobic environment of the infrainfundibulum, where it releases lipases and digests local accumulations of the skin, oil and sebum. Sebaceous glands produce an oily sebum that is primarily composed of waxes, triglycerides, and free fatty acids. (Lu et al., “Comparison of artificial sebum with human and hamster sebum samples,” Int. J. Pharm., (Epub date, Oct. 22, 2008); Valiveti et al., “Diffusion properties of model compounds in artificial sebum,” Int. J. Pharm., 345:88-94 (2007); and Valiveti et al., “Investigation of drug partition property in artificial sebum,” Int. J. Pharm., 346:10-16 (2008).) Studies described herein have shown that nanoemulsion droplets of the compositions described herein (NB-00X nanodroplets) are concentrated in the pilosebaceous unit where P. acnes migrates to enjoy a rich source of food (sebum) and a preferred anaerobic environment. (Ciotti et al., “Novel nanoemulsion NB-001 permeates skin by the follucular route. Abstr. 48th Intersci. Conf. on Antimicrob. Agents Chemother., abstr. A-1898 (2008).)

One effect that impacts acne prevention and/or treatment is the reduction of P. acnes. Specifically, this anti-acne effect can be expressed in vitro as minimum inhibitory concentration (MIC) and minimum bactericidal concentration (MBC) values, of a nanoemulsion of the invention and compared to the effect of other anti-acne drugs currently used for the treatment of acne, on different strains of P. acnes. Surprisingly, the comparison shows that the nanoemulsions of the invention are active against P. acnes, including antibiotic-resistant strains. The minimum inhibitory concentrations (MIC₉₀) and minimum bactericidal concentrations (MBC₉₀) for 90% of the isolates were 0.5 μg/ml/2.0 μg/ml for NB-00X and 1 μg/ml/2 μg/ml for NB-00X gel, respectively. Greater than 50% of the isolates were resistant to erythromycin and clindamycin; 44% of the isolates were resistant to tetracycline. If the MBC₉₀/MIC₉₀ ratio is the agent is bactericidal; if >4, the agent is bacteriostatic.

Example 5 below details the efficacy of a nanoemulsion according to the invention against Propionibacterium acnes in the presence of artificial sebum. In particular, as shown in Example 5, the MICs of a nanoemulsion according to the invention without any additional EDTA showed a 32 to 64 fold increase in the presence of 25% artificial sebum. In addition, MBCs of a nanoemulsion according to the invention showed 256 fold increases in the presence of sebum. The addition of 10-20 mM of EDTA decreased the MICs and MBCs of a nanoemulsion according to the invention to equal or lesser than the test concentrations.

The nanoemulsions comprise droplets having an average diameter of less than about 3 microns, and the nanoemulsions comprise an aqueous phase, at least one oil, at least one surfactant or detergent, and at least one organic solvent. In one embodiment of the invention, the surfactant present in the nanoemulsion is a cationic surfactant. More than one surfactant or detergent can be present in the nanoemulsions of the invention, and the second surfactant can be the same type (i.e., two cationic surfactants) or the second or third etc. surfactant can be different from the first. For example, the nanoemulsions can comprise a cationic surfactant in combination with a non-ionic surfactant. In another embodiment of the invention, the nanoemulsion further comprises a chelating agent. The organic solvent and the aqueous phase of the invention can be a non-phosphate based solvent.

In some embodiments, a second anti-acne agent is also incorporated into the nanoemulsion to achieve improved delivery, efficacy and/or tolerability of the added anti-acne agent. Examples of suitable topical anti-acne agents include, but are not limited to, benzoyl peroxide, salicylic acid, acitretin, alcloxa, aldioxa, allantoin, dibenzothiophene, etarotent, etretinate, motretinide, nordihydroguaiaretic acid, podofilox, podophyllum resin, resorcinalm resorcinol monoacetate, sumarotene, tetroquinone, tetracycline, doxycycline, minocycline, meclocycline erythromycin, clindamycin, azelaic acid, hydrocortisone, sodium hyaluronate, sulfur, urea, dapsone, adapalene, tretinoin, retinoids and retinoid-derived compounds. Other anti-acne ingredients include Ascorbyl Tetraisopalmitate, Dipotassium Glycyrrhizinate, Ascorbyl Tetraisopalmitate, Niacinamide, alpha bisabolol. All of these skin care ingredients have properties that help to reduce and control acne, and acne related problems such as sebum production. Examples of acne herbal medicines include, but are not limited to, Tea Tree Oil red clover, lavender, leaves of strawberry, chaste tree berry extract, burdock root, dandelion leaves, milk thistle, papaya enzymes, burdock and dandelion, eucalyptus, thyme, witch hazel, sage oil, camphor, cineole, rosmarinic acid and tannins in the sage oil.

The nanoemulsions comprise high energy nanometer-sized droplets that permeate into the pilosebaceous unit where they kill or inhibit the growth of P. acnes. Droplets having a suitable particle size can permeate skin pores and into the pilosebaceous unit, but can be excluded by tight junctions between epithelial cells and thus do not disrupt tissue matrices or enter blood vessels. This minimizes skin irritation and systemic absorption, but yet provides for a composition which is highly topically bioavailable in the pilosebaceous unit, epidermal and dermal tissues without causing disruption to the normal epithelial matrix.

In one embodiment of the invention, the nanoemulsion comprises: (a) an aqueous phase; (b) about 1% oil to about 80% oil; (c) about 0.1% organic solvent to about 50% organic solvent; (d) about 0.001% surfactant or detergent to about 10% surfactant or detergent; (e) about 0.0005% to about 1.0% of a chelating agent; or (0 any combination thereof. In another embodiment of the invention, the nanoemulsion comprises: (a) about 10% oil to about 80% oil; (b) about 1% organic solvent to about 50% organic solvent; (c) at least one non-ionic surfactant present in an amount of about 0.1% to about 10%; (d) at least one cationic agent present in an amount of about 0.01% to about 2%; (e) about 0.0005% to about 1.0% of a chelating agent; or (0 any combination thereof.

In yet another embodiment of the invention, the nanoemulsion additionally includes at least one suitable or desirable active agent useful in treating acne. The exemplary active agents for treating acne are benzoyl peroxide, salicylic acid and retinoids. The active agent can be present in a therapeutically effective amount, such as from about 0.001% up to about 99%, about 0.01% up to about 95%, about 0.1% up to about 90%, about 3% up to about 80%, about 5% up to about 60%, about 10% up to about 50%, or any combination thereof (e.g., about 3% up to about 10%).

The quantities of each component present in the nanoemulsion refer to a therapeutic nanoemulsion, and not to a nanoemulsion to be tested in vitro. This is significant, as nanoemulsions tested in vitro, such as the nanoemulsions described in the examples, generally have lower concentrations of oil, organic solvent, surfactant or detergent, and (if present) chelating agent than that present in a nanoemulsion intended for therapeutic use, e.g., topical use. This is because in vitro microbiology studies do not require the nanoemulsion droplets to traverse the skin or other barriers. For topical use, the concentrations of the components must be higher to result in therapeutic levels of nanoemulsion. However, the relative quantities of each component used in a nanoemulsion tested in vitro are applicable to a nanoemulsion to be used therapeutically and, therefore, in vitro quantities can be scaled up to prepare a therapeutic composition, and in vitro data may well be predictive of topical application success.

Viscosity

Examples 6 and 7 below demonstrate that increasing the viscosity of the nanoemulsion can enhance permeation of the nanoemulsion into the skin, thereby producing a nanoemulsion more effective in killing bacteria or other organisms.

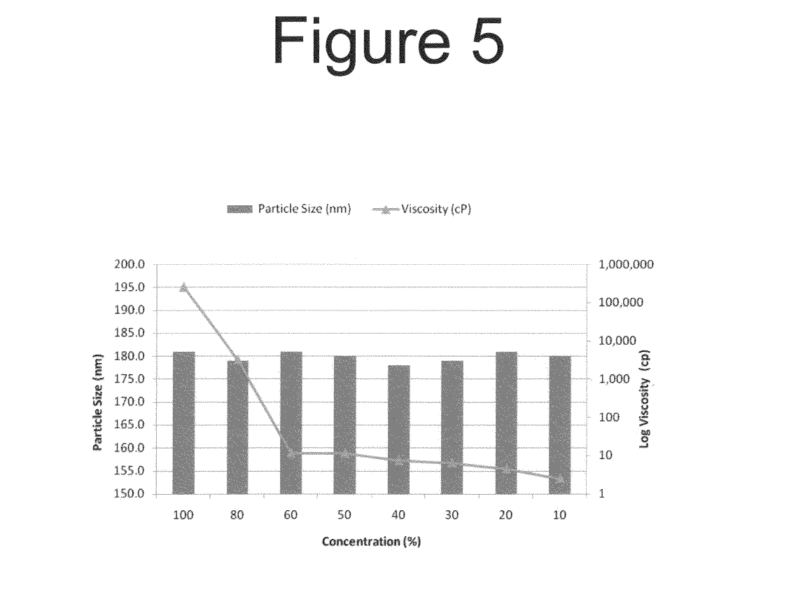

FIG. 5 shows the relationship between the particle size (nm), concentration of active (%), and viscosity of a nanoemulsion. The particle size does not change upon dilution of a nanoemulsion; however viscosity significantly decreases as a function of the decrease in particle concentrations. Thus, embodiment of the invention encompass using dilutions of a nanoemulsion. Table 14 (below) shows the effect dilution of a nanoemulsion has on the concentration of the active (CPC), viscosity, and particle size.

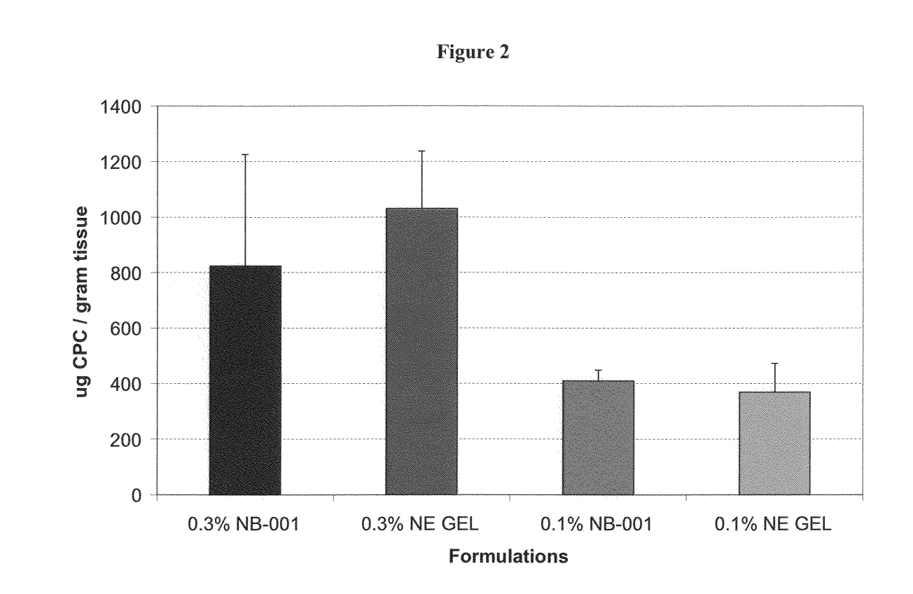

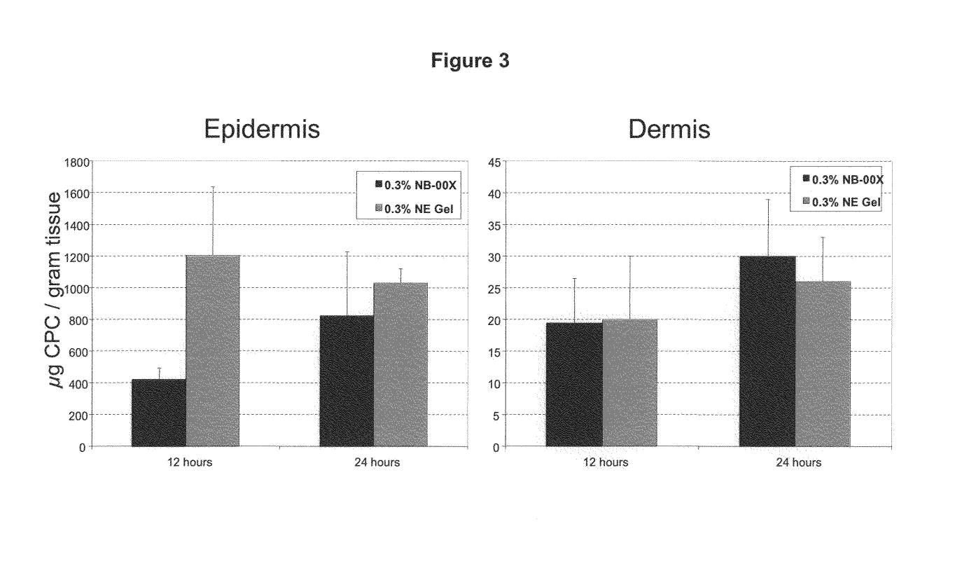

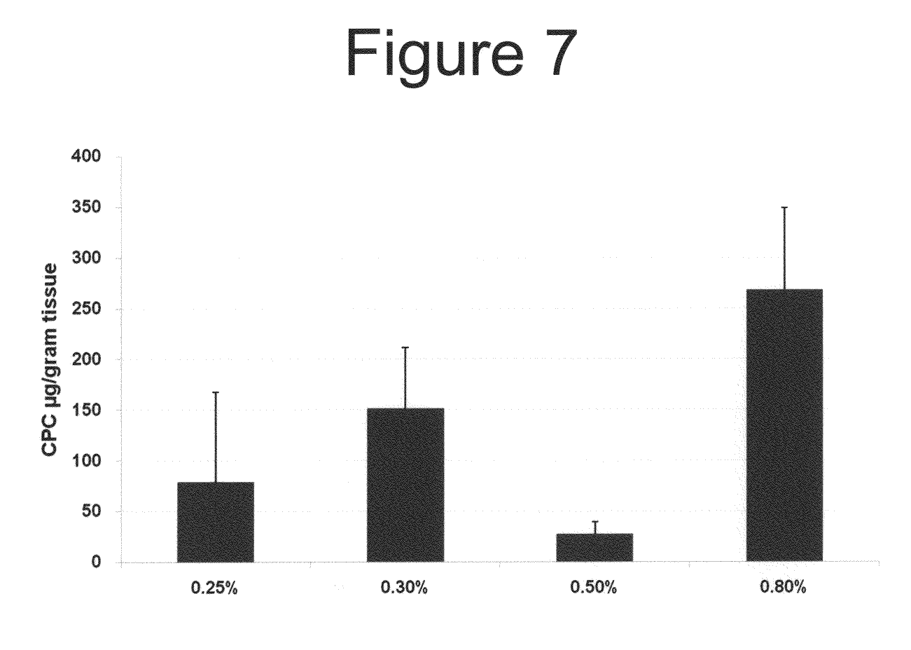

FIGS. 2, 3, 6 and 7 show the results for epidermis and dermis permeation, respectively. Higher viscosity nanoemulsions were found to increase the permeation of the nanoemulsion into the epidermis (FIGS. 2, 3 and FIG. 6) and dermis (FIGS. 3 and 7).

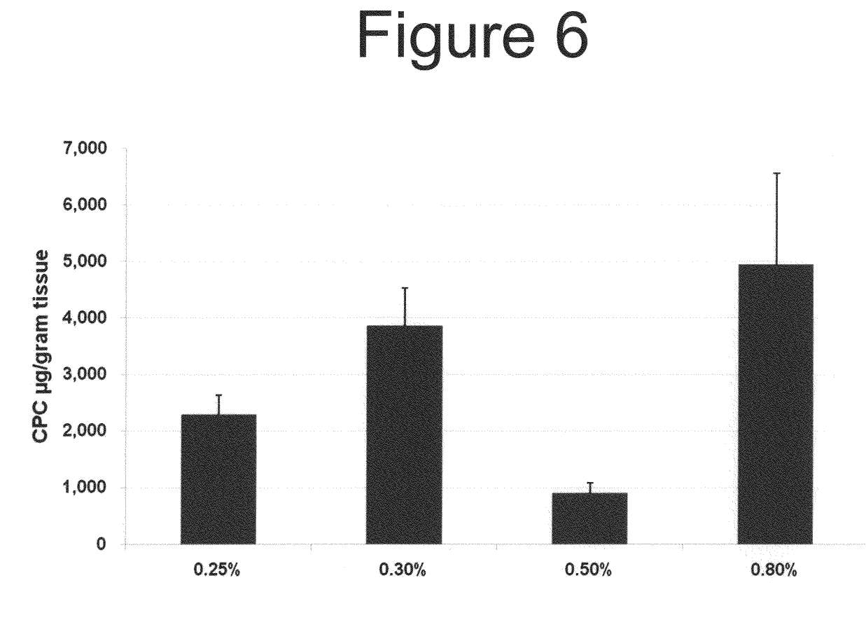

More particularly, as shown in FIGS. 6 and 7, lower concentration nanoemulsions, e.g., 0.25% to 0.30%, are effective in penetrating the skin. Slightly higher or lower concentrations are also effective. However, at a concentration of 0.5%, permeation significantly declined. Surprisingly, higher concentrations such as 0.8% or more showed a dramatic increase in permeation due to the increased viscosity of the composition. It is theorized that the increase in viscosity inhibits or limits the evaporation of water from the skin after application of the emulsion, thus preventing the crystallization of the active from the nanoemulsion. As an alternative to increasing the concentration of the nanoemulsion, the viscosity of the nanoemulsion can be increased to provide improved therapeutic effectiveness. Examples of methods of increasing the viscosity of a nanoemulsion according to the invention including increasing the concentration of the nanoemulsion (e.g., increasing CPC concentration), or adding a thickening agent or gelling agent to the formulation (see e.g., FIGS. 2 and 3).

Thus, in one embodiment of the invention, the nanoemulsion has a viscosity of greater than about 12 centipoise (cP), greater than about 15 cP, greater than about 20 cP, greater than about 25 cP, greater than about 30 cP, greater than about 35 cP, greater than about 40 cP, greater than about 45 cP, greater than about 50 cP, greater than about 55 cP, greater than about 60 cP, greater than about 65 cP, greater than about 70 cP, greater than about 75 cP, greater than about 80 cP, greater than about 85 cP, greater than about 90 cP, greater than about 95 cP, greater than about 100 cP, greater than about 150 cP, greater than about 200 cP, greater than about 300 cP, greater than about 400 cP, greater than about 500 cP, greater than about 600 cP, greater than about 700 cP, greater than about 800 cP, greater than about 900 cP, greater than about 1000 cP, greater than about 1500 cP, greater than about 2000 cP, greater than about 2500 cP, greater than about 3000 cP, greater than about 3500 cP, greater than about 4000 cP, greater than about 4500 cP, greater than about 5000 cP, greater than about 5500 cP, greater than about 6000 cP, greater than about 7000 cP, greater than about 8000 cP, greater than about 9000 cP, greater than about 10,000 cP, greater than about 15,000 cP, greater than about 20,000 cP, greater than about 30,000 cP, greater than about 40,000 cP, greater than about 50,000 cP, greater than about 60,000 cP, greater than about 70,000 cP, greater than about 80,000 cP, greater than about 90,000 cP, greater than about 100,000 cP, greater than about 150,000 cP, greater than about 200,000 cP, greater than about 250,000 cP, or up to about 259,300 cP.

Temperature

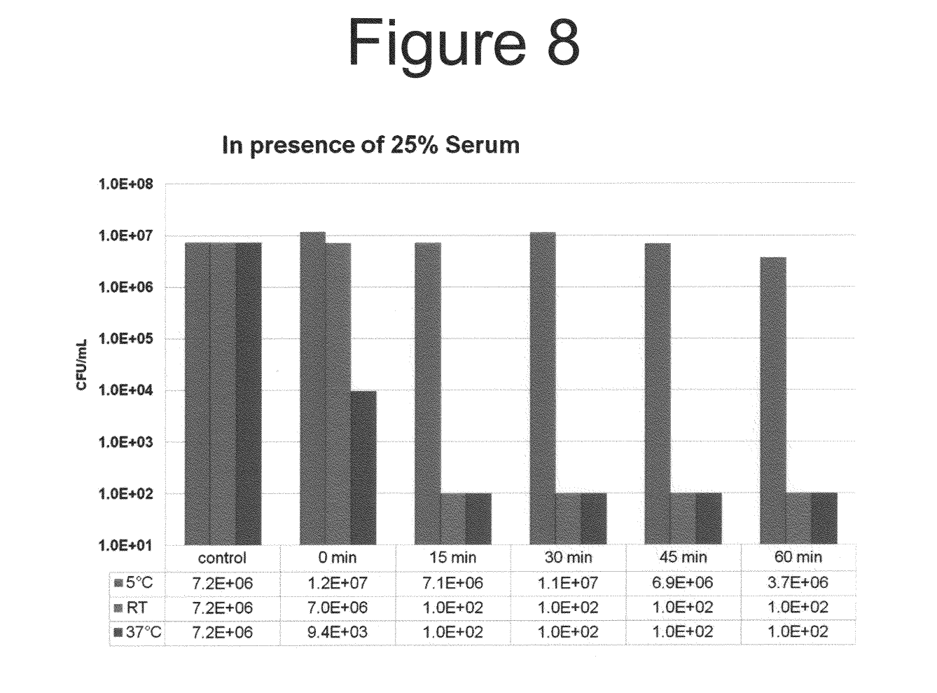

As described in Example 8, one tactic that can increase the effectiveness of a nanoemulsion according to the invention in treating acne is ensuring that the nanoemulsion is at room temperature or warmer prior to application. The results of Example 8, depicted in FIG. 8, show that cooling the nanoemulsion decreases the effectiveness of the nanoemulsion in killing P. acnes. Conversely, nanoemulsions at room temperature and warmed to 37° C. showed an increased effectiveness in killing P. acnes. The nanoemulsion warmed to 37° C. showed an initial greater effectiveness in killing P. acnes as compared to the room temperature nanoemulsion, with this increase in effectiveness diminishing about 15 minutes after application.

Thus, in another embodiment of the invention, encompassed are methods of treating acne comprising application of a nanoemulsion according to the invention, wherein the nanoemulsion is at room temperature (e.g., 20 to 25° C.). In another embodiment of the invention, encompassed are methods of treating acne comprising application of a nanoemulsion according to the invention, wherein the nanoemulsion has been warmed prior to application. For example, the nanoemulsion can be warmed prior to application to a temperature selected from the group consisting of about 30° C. or warmer, about 31° C. or warmer, about 32° C. or warmer, about 33° C. or warmer, about 34° C. or warmer, about 35° C. or warmer, about 36° C. or warmer, about 37° C. or warmer,

### A. DEFINITIONS

The present invention is described herein using several definitions, as set forth below and throughout the application.

As used herein, “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

The terms “buffer” or “buffering agents” refer to materials which when added to a solution, cause the solution to resist changes in pH.

The terms “chelator” or “chelating agent” refer to any materials having more than one atom with a lone pair of electrons that are available to bond to a metal ion.

The term “dilution” refers to dilution of the nanoemulsions of the present invention or those derived from the nanoemulsions of the present invention using, for example, an aqueous system comprised of PBS or water (such as diH₂O), or other water soluble components, to the desired final concentration.

The term “nanoemulsion,” as used herein, includes dispersions or droplets, as well as other lipid structures that can form as a result of hydrophobic forces that drive apolar residues (i.e., long hydrocarbon chains) away from water and drive polar head groups toward water, when a water immiscible oily phase is mixed with an aqueous phase. These other lipid structures include, but are not limited to, unilamellar, paucilamellar, and multilamellar lipid vesicles, micelles, and lamellar phases. The droplets have an average diameter of less than about 3 microns.

The terms “pharmaceutically acceptable” or “pharmacologically acceptable,” as used herein, refer to compositions that do not substantially produce adverse allergic or immunological reactions when administered to a host (e.g., an animal or a human). Such formulations include any pharmaceutically acceptable dosage form. As used herein, “pharmaceutically acceptable carrier” includes any and all solvents, dispersion media, coatings, wetting agents (e.g., sodium lauryl sulfate), isotonic and absorption delaying agents, disintegrants (e.g., potato starch or sodium starch glycolate), and the like.

The term “stable” when referring to a “stable nanoemulsion” means that the nanoemulsion retains its structure as an emulsion. A desired nanoemulsion structure, for example, may be characterized by a desired size range, macroscopic observations of emulsion science (is there one or more layers visible, is there visible precipitate), pH, and a stable concentration of one or more the components.

The term “subject” as used herein refers to organisms to be treated by the compositions of the present invention. Such organisms include animals (domesticated animal species, wild animals), and humans.

The term “surfactant” refers to any molecule having both a polar head group, which energetically prefers solvation by water, and a hydrophobic tail which is not well solvated by water. The term “cationic surfactant” refers to a surfactant with a cationic head group. The term “anionic surfactant” refers to a surfactant with an anionic head group.

As used herein, the term “topically” refers to application of the compositions of the present invention to the surface of the skin and tissues.

### B. STABILITY OF THE NANOEMULSIONS OF THE INVENTION

The nanoemulsions of the invention are stable at about 40° C. and about 75% relative humidity for a time period of at least up to about 1 month, at least up to about 3 months, at least up to about 6 months, at least up to about 12 months, at least up to about 18 months, at least up to about 2 years, at least up to about 2.5 years, or at least up to about 3 years.

In another embodiment of the invention, the nanoemulsions of the invention are stable at about 25° C. and about 60% relative humidity for a time period of at least up to about 1 month, at least up to about 3 months, at least up to about 6 months, at least up to about 12 months, at least up to about 18 months, at least up to about 2 years, at least up to about 2.5 years, or at least up to about 3 years, at least up to about 3.5 years, at least up to about 4 years, at least up to about 4.5 years, or at least up to about 5 years.

Further, the nanoemulsions of the invention are stable at about 4° C. for a time period of at least up to about 1 month, at least up to about 3 months, at least up to about 6 months, at least up to about 12 months, at least up to about 18 months, at least up to about 2 years, at least up to about 2.5 years, at least up to about 3 years, at least up to about 3.5 years, at least up to about 4 years, at least up to about 4.5 years, at least up to about 5 years, at least up to about 5.5 years, at least up to about 6 years, at least up to about 6.5 years, or at least up to about 7 years.

### C. NANOEMULSIONS

The term “nanoemulsion”, as defined herein, refers to a dispersion or droplet or any other lipid structure. Typical lipid structures contemplated in the invention include, but are not limited to, unilamellar, paucilamellar and multilamellar lipid vesicles, micelles and lamellar phases.

The nanoemulsion of the present invention comprises droplets having an average diameter size of less than about 3 microns, less than about 2500 nm, less than about 2000 nm, less than about 1500 nm, less than about 1000 nm, less than about 950 nm, less than about 900 nm, less than about 850 nm, less than about 800 nm, less than about 750 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 200 nm, less than about 150 nm, or any combination thereof. In one embodiment, the droplets have an average diameter size greater than about 125 nm and at least 400 nm. In another embodiment, the droplets have an average diameter of 180 nm.

1. Aqueous Phase

The aqueous phase can comprise any type of aqueous phase including, but not limited to, water (e.g., H₂O, distilled water, tap water) and solutions (e.g., phosphate-buffered saline (PBS) solution). In certain embodiments, the aqueous phase comprises water at a pH of about 4 to 10, preferably about 6 to 8. The water can be deionized (hereinafter “DiH₂O”). In some embodiments the aqueous phase comprises phosphate-buffered saline (PBS). The aqueous phase may further be sterile and pyrogen free.

2. Organic Solvents

Organic solvents in the nanoemulsions of the invention include, but are not limited to, C₁-C₁₂ alcohol, diol, triol, dialkyl phosphate, tri-alkyl phosphate, such as tri-n-butyl phosphate, semi-synthetic derivatives thereof, and combinations thereof. In one aspect of the invention, the organic solvent is an alcohol chosen from a nonpolar solvent, a polar solvent, a protic solvent, or an aprotic solvent.

Suitable organic solvents for the nanoemulsion include, but are not limited to, ethanol, methanol, isopropyl alcohol, glycerol, medium chain triglycerides, diethyl ether, ethyl acetate, acetone, dimethyl sulfoxide (DMSO), acetic acid, n-butanol, butylene glycol, perfumers alcohols, isopropanol, n-propanol, formic acid, propylene glycols, glycerol, sorbitol, industrial methylated spirit, triacetin, hexane, benzene, toluene, diethyl ether, chloroform, 1,4-dixoane, tetrahydrofuran, dichloromethane, acetone, acetonitrile, dimethylformamide, dimethyl sulfoxide, formic acid, semi-synthetic derivatives thereof, and any combination thereof.

3. Oil Phase

The oil in the nanoemulsion of the invention can be any cosmetically or pharmaceutically acceptable oil. The oil can be volatile or non-volatile, and may be chosen from animal oil, vegetable oil, natural oil, synthetic oil, hydrocarbon oils, silicone oils, semi-synthetic derivatives thereof, and combinations thereof.

Suitable oils include, but are not limited to, mineral oil, squalene oil, flavor oils, silicon oil, essential oils, water insoluble vitamins, Isopropyl stearate, Butyl stearate, Octyl palmitate, Cetyl palmitate, Tridecyl behenate, Diisopropyl adipate, Dioctyl sebacate, Menthyl anthranhilate, Cetyl octanoate, Octyl salicylate, Isopropyl myristate, neopentyl glycol dicarpate cetols, Ceraphyls®, Decyl oleate, diisopropyl adipate, C₁₂₋₁₅ alkyl lactates, Cetyl lactate, Lauryl lactate, Isostearyl neopentanoate, Myristyl lactate, Isocetyl stearoyl stearate, Octyldodecyl stearoyl stearate, Hydrocarbon oils, Isoparaffin, Fluid paraffins, Isododecane, Petrolatum, Argan oil, Canola oil, Chile oil, Coconut oil, corn oil, Cottonseed oil, Flaxseed oil, Grape seed oil, Mustard oil, Olive oil, Palm oil, Palm kernel oil, Peanut oil, Pine seed oil, Poppy seed oil, Pumpkin seed oil, Rice bran oil, Safflower oil, Tea oil, Truffle oil, Vegetable oil, Apricot (kernel) oil, Jojoba oil (simmondsia chinensis seed oil), Grapeseed oil, Macadamia oil, Wheat germ oil, Almond oil, Rapeseed oil, Gourd oil, Soybean oil, Sesame oil, Hazelnut oil, Maize oil, Sunflower oil, Hemp oil, Bois oil, Kuki nut oil, Avocado oil, Walnut oil, Fish oil, berry oil, allspice oil, juniper oil, seed oil, almond seed oil, anise seed oil, celery seed oil, cumin seed oil, nutmeg seed oil, leaf oil, basil leaf oil, bay leaf oil, cinnamon leaf oil, common sage leaf oil, eucalyptus leaf oil, lemon grass leaf oil, melaleuca leaf oil, oregano leaf oil, patchouli leaf oil, peppermint leaf oil, pine needle oil, rosemary leaf oil, spearmint leaf oil, tea tree leaf oil, thyme leaf oil, wintergreen leaf oil, flower oil, chamomile oil, clary sage oil, clove oil, geranium flower oil, hyssop flower oil, jasmine flower oil, lavender flower oil, manuka flower oil, Marhoram flower oil, orange flower oil, rose flower oil, ylang-ylang flower oil, Bark oil, cassia Bark oil, cinnamon bark oil, sassafras Bark oil, Wood oil, camphor wood oil, cedar wood oil, rosewood oil, sandalwood oil), rhizome (ginger) wood oil, resin oil, frankincense oil, myrrh oil, peel oil, bergamot peel oil, grapefruit peel oil, lemon peel oil, lime peel oil, orange peel oil, tangerine peel oil, root oil, valerian oil, Oleic acid, Linoleic acid, Oleyl alcohol, Isostearyl alcohol, semi-synthetic derivatives thereof, and any combinations thereof.

The oil may further comprise a silicone component, such as a volatile silicone component, which can be the sole oil in the silicone component or can be combined with other silicone and non-silicone, volatile and non-volatile oils. Suitable silicone components include, but are not limited to, methylphenylpolysiloxane, simethicone, dimethicone, phenyltrimethicone (or an organomodified version thereof), alkylated derivatives of polymeric silicones, cetyl dimethicone, lauryl trimethicone, hydroxylated derivatives of polymeric silicones, such as dimethiconol, volatile silicone oils, cyclic and linear silicones, cyclomethicone, derivatives of cyclomethicone, hexamethylcyclotrisiloxane, octamethylcyclotetrasiloxane, decamethylcyclopentasiloxane, volatile linear dimethylpolysiloxanes, isohexadecane, isoeicosane, isotetracosane, polyisobutene, isooctane, isododecane, semi-synthetic derivatives thereof, and combinations thereof.

The volatile oil can be the organic solvent, or the volatile oil can be present in addition to an organic solvent. Suitable volatile oils include, but are not limited to, a terpene, monoterpene, sesquiterpene, carminative, azulene, menthol, camphor, thujone, thymol, nerol, linalool, limonene, geraniol, perillyl alcohol, nerolidol, farnesol, ylangene, bisabolol, farnesene, ascaridole, chenopodium oil, citronellal, citral, citronellol, chamazulene, yarrow, guaiazulene, chamomile, semi-synthetic derivatives, or combinations thereof.

In one aspect of the invention, the volatile oil in the silicone component is different than the oil in the oil phase.

4. Surfactants/Detergent

The surfactant or detergent in the nanoemulsion of the invention can be a pharmaceutically acceptable ionic surfactant, a pharmaceutically acceptable nonionic surfactant, a pharmaceutically acceptable cationic surfactant, a pharmaceutically acceptable anionic surfactant, or a pharmaceutically acceptable zwitterionic surfactant.

Exemplary useful surfactants are described in Applied Surfactants: Principles and Applications. Tharwat F. Tadros, Copyright 8 2005 WILEY-VCH Verlag GmbH & Co. KGaA, Weinheim ISBN: 3-527-30629-3), which is specifically incorporated by reference.

Further, the surfactant can be a pharmaceutically acceptable ionic polymeric surfactant, a pharmaceutically acceptable nonionic polymeric surfactant, a pharmaceutically acceptable cationic polymeric surfactant, a pharmaceutically acceptable anionic polymeric surfactant, or a pharmaceutically acceptable zwitterionic polymeric surfactant. Examples of polymeric surfactants include, but are not limited to, a graft copolymer of a poly(methyl methacrylate) backbone with multiple (at least one) polyethylene oxide (PEO) side chain, polyhydroxystearic acid, an alkoxylated alkyl phenol formaldehyde condensate, a polyalkylene glycol modified polyester with fatty acid hydrophobes, a polyester, semi-synthetic derivatives thereof, or combinations thereof.

Surface active agents or surfactants, are amphipathic molecules that consist of a non-polar hydrophobic portion, usually a straight or branched hydrocarbon or fluorocarbon chain containing 8-18 carbon atoms, attached to a polar or ionic hydrophilic portion. The hydrophilic portion can be nonionic, ionic or zwitterionic. The hydrocarbon chain interacts weakly with the water molecules in an aqueous environment, whereas the polar or ionic head group interacts strongly with water molecules via dipole or ion-dipole interactions. Based on the nature of the hydrophilic group, surfactants are classified into anionic, cationic, zwitterionic, nonionic and polymeric surfactants.

Suitable surfactants include, but are not limited to, ethoxylated nonylphenol comprising 9 to 10 units of ethyleneglycol, ethoxylated undecanol comprising 8 units of ethyleneglycol, polyoxyethylene (20) sorbitan monolaurate, polyoxyethylene (20) sorbitan monopalmitate, polyoxyethylene (20) sorbitan monostearate, polyoxyethylene (20) sorbitan monooleate, sorbitan monolaurate, sorbitan monopalmitate, sorbitan monostearate, sorbitan monooleate, ethoxylated hydrogenated ricin oils, sodium laurylsulfate, a diblock copolymer of ethyleneoxyde and propyleneoxyde, Ethylene Oxide-Propylene Oxide Block Copolymers, and tetra-functional block copolymers based on ethylene oxide and propylene oxide, Glyceryl monoesters, Glyceryl caprate, Glyceryl caprylate, Glyceryl cocate, Glyceryl erucate, Glyceryl hydroxysterate, Glyceryl isostearate, Glyceryl lanolate, Glyceryl laurate, Glyceryl linolate, Glyceryl myristate, Glyceryl oleate, Glyceryl PABA, Glyceryl palmitate, Glyceryl ricinoleate, Glyceryl stearate, Glyceryl thighlycolate, Glyceryl dilaurate, Glyceryl dioleate, Glyceryl dimyristate, Glyceryl disterate, Glyceryl sesuioleate, Glyceryl stearate lactate, Polyoxyethylene cetyl/stearyl ether, Polyoxyethylene cholesterol ether, Polyoxyethylene laurate or dilaurate, Polyoxyethylene stearate or distearate, polyoxyethylene fatty ethers, Polyoxyethylene lauryl ether, Polyoxyethylene stearyl ether, polyoxyethylene myristyl ether, a steroid, Cholesterol, Betasitosterol, Bisabolol, fatty acid esters of alcohols, isopropyl myristate, Aliphati-isopropyl n-butyrate, Isopropyl n-hexanoate, Isopropyl n-decanoate, Isoproppyl palmitate, Octyldodecyl myristate, alkoxylated alcohols, alkoxylated acids, alkoxylated amides, alkoxylated sugar derivatives, alkoxylated derivatives of natural oils and waxes, polyoxyethylene polyoxypropylene block copolymers, nonoxynol-14, PEG-8 laurate, PEG-6 Cocoamide, PEG-20 methylglucose sesquistearate, PEG40 lanolin, PEG-40 castor oil, PEG-40 hydrogenated castor oil, polyoxyethylene fatty ethers, glyceryl diesters, polyoxyethylene stearyl ether, polyoxyethylene myristyl ether, and polyoxyethylene lauryl ether, glyceryl dilaurate, glyceryl dimystate, glyceryl distearate, semi-synthetic derivatives thereof, or mixtures thereof.

Additional suitable surfactants include, but are not limited to, non-ionic lipids, such as glyceryl laurate, glyceryl myristate, glyceryl dilaurate, glyceryl dimyristate, semi-synthetic derivatives thereof, and mixtures thereof.

In additional embodiments, the surfactant is a polyoxyethylene fatty ether having a polyoxyethylene head group ranging from about 2 to about 100 groups, or an alkoxylated alcohol having the structure R₅—(OCH₂CH₂)_{y}—OH, wherein R₅ is a branched or unbranched alkyl group having from about 6 to about 22 carbon atoms and y is between about 4 and about 100, and preferably, between about 10 and about 100. Preferably, the alkoxylated alcohol is the species wherein R₅ is a lauryl group and y has an average value of 23.

In a different embodiment, the surfactant is an alkoxylated alcohol which is an ethoxylated derivative of lanolin alcohol. Preferably, the ethoxylated derivative of lanolin alcohol is laneth-10, which is the polyethylene glycol ether of lanolin alcohol with an average ethoxylation value of 10.

Nonionic surfactants include, but are not limited to, an ethoxylated surfactant, an alcohol ethoxylated, an alkyl phenol ethoxylated, a fatty acid ethoxylated, a monoalkaolamide ethoxylated, a sorbitan ester ethoxylated, a fatty amino ethoxylated, an ethylene oxide-propylene oxide copolymer, Bis(polyethylene glycol bis[imidazoyl carbonyl]), nonoxynol-9, Bis(polyethylene glycol bis[imidazoyl carbonyl]), Brij® 35, Brij® 56, Brij® 72, Brij® 76, Brij® 92V, Brij® 97, Brij® 58P, Cremophor® EL, Decaethylene glycol monododecyl ether, N-Decanoyl-N-methylglucamine, n-Decyl alpha-D-glucopyranoside, Decyl beta-D-maltopyranoside, n-Dodecanoyl-N-methylglucamide, n-Dodecyl alpha-D-maltoside, n-Dodecyl beta-D-maltoside, n-Dodecyl beta-D-maltoside, Heptaethylene glycol monodecyl ether, Heptaethylene glycol monododecyl ether, Heptaethylene glycol monotetradecyl ether, n-Hexadecyl beta-D-maltoside, Hexaethylene glycol monododecyl ether, Hexaethylene glycol monohexadecyl ether, Hexaethylene glycol monooctadecyl ether, Hexaethylene glycol monotetradecyl ether, Igepal CA-630, Igepal CA-630, Methyl-6-O-(N-heptylcarbamoyl)-alpha-D-glucopyranoside, Nonaethylene glycol monododecyl ether, N-N-Nonanoyl-N-methylglucamine, Octaethylene glycol monodecyl ether, Octaethylene glycol monododecyl ether, Octaethylene glycol monohexadecyl ether, Octaethylene glycol monooctadecyl ether, Octaethylene glycol monotetradecyl ether, Octyl-beta-D-glucopyranoside, Pentaethylene glycol monodecyl ether, Pentaethylene glycol monododecyl ether, Pentaethylene glycol monohexadecyl ether, Pentaethylene glycol monohexyl ether, Pentaethylene glycol monooctadecyl ether, Pentaethylene glycol monooctyl ether, Polyethylene glycol diglycidyl ether, Polyethylene glycol ether W-1, Polyoxyethylene 10 tridecyl ether, Polyoxyethylene 100 stearate, Polyoxyethylene 20 isohexadecyl ether, Polyoxyethylene 20 oleyl ether, Polyoxyethylene 40 stearate, Polyoxyethylene 50 stearate, Polyoxyethylene 8 stearate, Polyoxyethylene bis(imidazolyl carbonyl), Polyoxyethylene 25 propylene glycol stearate, Saponin from Quillaja bark, Span® 20, Span® 40, Span® 60, Span® 65, Span® 80, Span® 85, Tergitol, Type 15-S-12, Tergitol, Type 15-S-30, Tergitol, Type 15-S-5, Tergitol, Type 15-S-7, Tergitol, Type 15-S-9, Tergitol, Type NP-10, Tergitol, Type NP-4, Tergitol, Type NP-40, Tergitol, Type NP-7, Tergitol, Type NP-9, Tergitol, Tergitol, Type TMN-10, Tergitol, Type TMN-6, Tetradecyl-beta-D-maltoside, Tetraethylene glycol monodecyl ether, Tetraethylene glycol monododecyl ether, Tetraethylene glycol monotetradecyl ether, Triethylene glycol monodecyl ether, Triethylene glycol monododecyl ether, Triethylene glycol monohexadecyl ether, Triethylene glycol monooctyl ether, Triethylene glycol monotetradecyl ether, Triton CF-21, Triton CF-32, Triton DF-12, Triton DF-16, Triton GR-5M, Triton QS-15, Triton QS-44, Triton X-100, Triton X-102, Triton X-15, Triton X-151, Triton X-200, Triton X-207, Triton® X-114, Triton® X-165, Triton® X-305, Triton® X-405, Triton® X-45, Triton® X-705-70, TWEEN® 20, TWEEN® 21, TWEEN® 40, TWEEN® 60, TWEEN® 61, TWEEN® 65, TWEEN® 80, TWEEN® 81, TWEEN® 85, Tyloxapol, n-Undecyl beta-D-glucopyranoside, semi-synthetic derivatives thereof, or combinations thereof.

In addition, the nonionic surfactant can be a poloxamer. Poloxamers are polymers made of a block of polyoxyethylene, followed by a block of polyoxypropylene, followed by a block of polyoxyethylene. The average number of units of polyoxyethylene and polyoxypropylene varies based on the number associated with the polymer. For example, the smallest polymer, Poloxamer 101, consists of a block with an average of 2 units of polyoxyethylene, a block with an average of 16 units of polyoxypropylene, followed by a block with an average of 2 units of polyoxyethylene. Poloxamers range from colorless liquids and pastes to white solids. In cosmetics and personal care products, Poloxamers are used in the formulation of skin cleansers, bath products, shampoos, hair conditioners, mouthwashes, eye makeup remover and other skin and hair products. Examples of Poloxamers include, but are not limited to, Poloxamer 101, Poloxamer 105, Poloxamer 108, Poloxamer 122, Poloxamer 123, Poloxamer 124, Poloxamer 181, Poloxamer 182, Poloxamer 183, Poloxamer 184, Poloxamer 185, Poloxamer 188, Poloxamer 212, Poloxamer 215, Poloxamer 217, Poloxamer 231, Poloxamer 234, Poloxamer 235, Poloxamer 237, Poloxamer 238, Poloxamer 282, Poloxamer 284, Poloxamer 288, Poloxamer 331, Poloxamer 333, Poloxamer 334, Poloxamer 335, Poloxamer 338, Poloxamer 401, Poloxamer 402, Poloxamer 403, Poloxamer 407, Poloxamer 105 Benzoate, and Poloxamer 182 Dibenzoate.

Suitable cationic surfactants include, but are not limited to, a quarternary ammonium compound, an alkyl trimethyl ammonium chloride compound, a dialkyl dimethyl ammonium chloride compound, a cationic halogen-containing compound, such as cetylpyridinium chloride, Benzalkonium chloride, Benzalkonium chloride, Benzyldimethylhexadecylammonium chloride, Benzyldimethyltetradecylammonium chloride, Benzyldodecyldimethylammonium bromide, Benzyltrimethylammonium tetrachloroiodate, Dimethyldioctadecylammonium bromide, Dodecylethyldimethylammonium bromide, Dodecyltrimethylammonium bromide, Dodecyltrimethylammonium bromide, Ethylhexadecyldimethylammonium bromide, Girard's reagent T, Hexadecyltrimethylammonium bromide, Hexadecyltrimethylammonium bromide, N,N′,N′-Polyoxyethylene(10)-N-tallow-1,3-diaminopropane, Thonzonium bromide, Trimethyl(tetradecyl)ammonium bromide, 1,3,5-Triazine-1,3,5(2H,4H,6H)-triethanol, 1-Decanaminium, N-decyl-N,N-dimethyl-, chloride, Didecyl dimethyl ammonium chloride, 2-(2-(p-(Diisobutyl)cresosxy)ethoxy)ethyl dimethyl benzyl ammonium chloride, 2-(2-(p-(Diisobutyl)phenoxy)ethoxy)ethyl dimethyl benzyl ammonium chloride, Alkyl 1 or 3 benzyl-1-(2-hydroxethyl)-2-imidazolinium chloride, Alkyl bis(2-hydroxyethyl)benzyl ammonium chloride, Alkyl demethyl benzyl ammonium chloride, Alkyl dimethyl 3,4-dichlorobenzyl ammonium chloride (100% C12), Alkyl dimethyl 3,4-dichlorobenzyl ammonium chloride (50% C14, 40% C12, 10% C16), Alkyl dimethyl 3,4-dichlorobenzyl ammonium chloride (55% C14, 23% C12, 20% C16), Alkyl dimethyl benzyl ammonium chloride, Alkyl dimethyl benzyl ammonium chloride (100% C14), Alkyl dimethyl benzyl ammonium chloride (100% C16), Alkyl dimethyl benzyl ammonium chloride (41% C14, 28% C12), Alkyl dimethyl benzyl ammonium chloride (47% C12, 18% C14), Alkyl dimethyl benzyl ammonium chloride (55% C16, 20% C14), Alkyl dimethyl benzyl ammonium chloride (58% C14, 28% C16), Alkyl dimethyl benzyl ammonium chloride (60% C14, 25% C12), Alkyl dimethyl benzyl ammonium chloride (61% C11, 23% C14), Alkyl dimethyl benzyl ammonium chloride (61% C12, 23% C14), Alkyl dimethyl benzyl ammonium chloride (65% C12, 25% C14), Alkyl dimethyl benzyl ammonium chloride (67% C12, 24% C14), Alkyl dimethyl benzyl ammonium chloride (67% C12, 25% C14), Alkyl dimethyl benzyl ammonium chloride (90% C14, 5% C12), Alkyl dimethyl benzyl ammonium chloride (93% C14, 4% C12), Alkyl dimethyl benzyl ammonium chloride (95% C16, 5% C18), Alkyl didecyl dimethyl ammonium chloride, Alkyl dimethyl benzyl ammonium chloride (C12-16), Alkyl dimethyl benzyl ammonium chloride (C12-18), dialkyl dimethyl benzyl ammonium chloride, Alkyl dimethyl dimethybenzyl ammonium chloride, Alkyl dimethyl ethyl ammonium bromide (90% C14, 5% C16, 5% C12), Alkyl dimethyl ethyl ammonium bromide (mixed alkyl and alkenyl groups as in the fatty acids of soybean oil), Alkyl dimethyl ethylbenzyl ammonium chloride, Alkyl dimethyl ethylbenzyl ammonium chloride (60% C14), Alkyl dimethyl isopropylbenzyl ammonium chloride (50% C12, 30% C14, 17% C16, 3% C18), Alkyl trimethyl ammonium chloride (58% C18, 40% C16, 1% C14, 1% C12), Alkyl trimethyl ammonium chloride (90% C18, 10% C16), Alkyldimethyl(ethylbenzyl) ammonium chloride (C12-18), Di-(C8-10)-alkyl dimethyl ammonium chlorides, Dialkyl dimethyl ammonium chloride, Dialkyl methyl benzyl ammonium chloride, Didecyl dimethyl ammonium chloride, Diisodecyl dimethyl ammonium chloride, Dioctyl dimethyl ammonium chloride, Dodecyl bis(2-hydroxyethyl) octyl hydrogen ammonium chloride, Dodecyl dimethyl benzyl ammonium chloride, Dodecylcarbamoyl methyl dimethyl benzyl ammonium chloride, Heptadecyl hydroxyethylimidazolinium chloride, Hexahydro-1,3,5-tris(2-hydroxyethyl)-s-triazine, Myristalkonium chloride (and) Quat RNIUM 14, N,N-Dimethyl-2-hydroxypropylammonium chloride polymer, n-Tetradecyl dimethyl benzyl ammonium chloride monohydrate, Octyl decyl dimethyl ammonium chloride, Octyl dodecyl dimethyl ammonium chloride, Octyphenoxyethoxyethyl dimethyl benzyl ammonium chloride, Oxydiethylenebis(alkyl dimethyl ammonium chloride), Trimethoxysily propyl dimethyl octadecyl ammonium chloride, Trimethoxysilyl quats, Trimethyl dodecylbenzyl ammonium chloride, semi-synthetic derivatives thereof, and combinations thereof.

Exemplary cationic halogen-containing compounds include, but are not limited to, cetylpyridinium halides, cetyltrimethylammonium halides, cetyldimethylethylammonium halides, cetyldimethylbenzylammonium halides, cetyltributylphosphonium halides, dodecyltrimethylammonium halides, or tetradecyltrimethylammonium halides. In some particular embodiments, suitable cationic halogen containing compounds comprise, but are not limited to, cetylpyridinium chloride (CPC), cetyltrimethylammonium chloride, cetylbenzyldimethylammonium chloride, cetylpyridinium bromide (CPB), cetyltrimethylammonium bromide (CTAB), cetyidimethylethylammonium bromide, cetyltributylphosphonium bromide, dodecyltrimethylammonium bromide, and tetrad ecyltrimethylammonium bromide. In particularly preferred embodiments, the cationic halogen containing compound is CPC, although the compositions of the present invention are not limited to formulation with a particular cationic containing compound.

Suitable anionic surfactants include, but are not limited to, a carboxylate, a sulphate, a sulphonate, a phosphate, chenodeoxycholic acid, chenodeoxycholic acid sodium salt, cholic acid, ox or sheep bile, Dehydrocholic acid, Deoxycholic acid, Deoxycholic acid, Deoxycholic acid methyl ester, Digitonin, Digitoxigenin, N,N-Dimethyldodecylamine N-oxide, Docusate sodium salt, Glycochenodeoxycholic acid sodium salt, Glycocholic acid hydrate, synthetic, Glycocholic acid sodium salt hydrate, synthetic, Glycodeoxycholic acid monohydrate, Glycodeoxycholic acid sodium salt, Glycolithocholic acid 3-sulfate disodium salt, Glycolithocholic acid ethyl ester, N-Lauroylsarcosine sodium salt, N-Lauroylsarcosine solution, N-Lauroylsarcosine solution, Lithium dodecyl sulfate, Lithium dodecyl sulfate, Lithium dodecyl sulfate, Lugol solution, Niaproof 4, Type 4,1-Octanesulfonic acid sodium salt, Sodium 1-butanesulfonate, Sodium 1-decanesulfonate, Sodium 1-decanesulfonate, Sodium 1-dodecanesulfonate, Sodium 1-heptanesulfonate anhydrous, Sodium 1-heptanesulfonate anhydrous, Sodium 1-nonanesulfonate, Sodium 1-propanesulfonate monohydrate, Sodium 2-bromoethanesulfonate, Sodium cholate hydrate, Sodium choleate, Sodium deoxycholate, Sodium deoxycholate monohydrate, Sodium dodecyl sulfate, Sodium hexanesulfonate anhydrous, Sodium octyl sulfate, Sodium pentanesulfonate anhydrous, Sodium taurocholate, Taurochenodeoxycholic acid sodium salt, Taurodeoxycholic acid sodium salt monohydrate, Taurohyodeoxycholic acid sodium salt hydrate, Taurolithocholic acid 3-sulfate disodium salt, Tauroursodeoxycholic acid sodium salt, Trizma® dodecyl sulfate, TWEEN® 80, Ursodeoxycholic acid, semi-synthetic derivatives thereof, and combinations thereof.

Suitable zwitterionic surfactants include, but are not limited to, an N-alkyl betaine, lauryl amindo propyl dimethyl betaine, an alkyl dimethyl glycinate, an N-alkyl amino propionate, CHAPS, minimum 98% (TLC), CHAPS, minimum 98% (TLC), CHAPS, for electrophoresis, minimum 98% (TLC), CHAPSO, minimum 98%, CHAPSO, CHAPSO, for electrophoresis, 3-(Decyldimethylammonio)propanesulfonate inner salt, 3-Dodecyldimethylammonio)propanesulfonate inner salt, 3-(Dodecyldimethylammonio)propanesulfonate inner salt, 3-(N,N-Dimethylmyristylammonio)propanesulfonate, 3-(N,N-Dimethyloctadecylammonio)propanesulfonate, 3-(N,N-Dimethyloctylammonio)propanesulfonate inner salt, 3-(N,N-Dimethylpalmitylammonio)propanesulfonate, semi-synthetic derivatives thereof, and combinations thereof.

In some embodiments, the nanoemulsion comprises a cationic surfactant, which can be cetylpyridinium chloride. In other embodiments of the invention, the nanoemulsion comprises a cationic surfactant, and the concentration of the cationic surfactant is less than about 5.0% and greater than about 0.001%. In yet another embodiment of the invention, the nanoemulsion comprises a cationic surfactant, and the concentration of the cationic surfactant is selected from the group consisting of less than about 5%, less than about 4.5%, less than about 4.0%, less than about 3.5%, less than about 3.0%, less than about 2.5%, less than about 2.0%, less than about 1.5%, less than about 1.0%, less than about 0.90%, less than about 0.80%, less than about 0.70%, less than about 0.60%, less than about 0.50%, less than about 0.40%, less than about 0.30%, less than about 0.20%, or less than about 0.10%. Further, the concentration of the cationic agent in the nanoemulsion is greater than about 0.002%, greater than about 0.003%, greater than about 0.004%, greater than about 0.005%, greater than about 0.006%, greater than about 0.007%, greater than about 0.008%, greater than about 0.009%, greater than about 0.010%, or greater than about 0.001%. In one embodiment, the concentration of the cationic agent in the nanoemulsion is less than about 5.0% and greater than about 0.001%.

In another embodiment of the invention, the nanoemulsion comprises at least one cationic surfactant and at least one non-cationic surfactant. The non-cationic surfactant is a nonionic surfactant, such as a polysorbate (Tween), such as polysorbate 80 or polysorbate 20. In one embodiment, the non-ionic surfactant is present in a concentration of about 0.05% to about 7.0%, or the non-ionic surfactant is present in a concentration of about 0.3% to about 4%. In yet another embodiment of the invention, the nanoemulsion comprises a cationic surfactant present in a concentration of about 0.01% to about 2%, in combination with a nonionic surfactant.

5. Active Agents

Optionally, a second anti-acne agent is incorporated into the nanoemulsion to achieve better efficacy, tolerability and/or synergistic antimicrobial activity effect in sebum. Preferably, the second anti-acne agent is benzoyl peroxide salicylic acid, or a retinoid. However, any active agent useful in treating acne can be incorporated into the nanoemulsion.

Exemplary topical anti-acne agents include, but are not limited to, benzoyl peroxide, salicylic acid, acitretin, alcloxa, aldioxa, allantoin, dibenzothiophene, etarotent, etretinate, motretinide, nordihydroguaiaretic acid, podofilox, podophyllum resin, resorcinalm resorcinol monoacetate, sumarotene, tetroquinone, adapalene, tretinoin, erythromycin, clindamycin, azelaic acid, hydrocortisone, sodium hyaluronate, sulfur, urea, meclocycline, dapsone, retinoids and retinoid derivatives. Other anti-acne ingredients include Ascorbyl Tetraisopalmitate, Dipotassium Glycyrrhizinate, Ascorbyl Tetraisopalmitate, Niacinamide, alpha bisabolol can also be included in the nanoemulsion of this invention. All of these skin care ingredients have properties that help to reduce and control acne, and acne related problems such as sebum production.

Additional anti-acne agents include acne herbal medicines, such as Tea Tree Oil red clover, lavender, leaves of strawberry, chaste tree berry extract, burdock root, dandelion leaves, milk thistle, papaya enzymes, burdock and dandelion, eucalyptus, thyme, witch hazel, sage oil, camphor, cineole, rosmarinic acid and tannins in the sage oil.

6. Additional Ingredients

Additional compounds suitable for use in the nanoemulsions of the invention include but are not limited to one or more solvents, such as an organic phosphate-based solvent, bulking agents, coloring agents, pharmaceutically acceptable excipients, a preservative, pH adjuster, buffer, chelating agent, etc. The additional compounds can be admixed into a previously emulsified nanoemulsion, or the additional compounds can be added to the original mixture to be emulsified. In certain of these embodiments, one or more additional compounds are admixed into an existing nanoemulsion composition immediately prior to its use.

Suitable preservatives in the nanoemulsions of the invention include, but are not limited to, cetylpyridinium chloride, benzalkonium chloride, benzyl alcohol, chlorhexidine, imidazolidinyl urea, phenol, potassium sorbate, benzoic acid, bronopol, chlorocresol, paraben esters, phenoxyethanol, sorbic acid, alpha-tocophernol, ascorbic acid, ascorbyl palmitate, butylated hydroxyanisole, butylated hydroxytoluene, sodium ascorbate, sodium metabisulphite, citric acid, edetic acid, semi-synthetic derivatives thereof, and combinations thereof.

The nanoemulsion may further comprise at least one pH adjuster. Suitable pH adjusters in the nanoemulsion of the invention include, but are not limited to, diethyanolamine, lactic acid, monoethanolamine, triethylanolamine, sodium hydroxide, sodium phosphate, semi-synthetic derivatives thereof, and combinations thereof.

In addition, the nanoemulsion can comprise a chelating agent. In one embodiment of the invention, the chelating agent is present in an amount of about 0.0005% to about 1.0%. Examples of chelating agents include, but are not limited to, ethylenediamine, ethylenediaminetetraacetic acid (EDTA), and dimercaprol, and a preferred chelating agent is ethylenediaminetetraacetic acid.

The nanoemulsion can comprise a buffering agent, such as a pharmaceutically acceptable buffering agent. Examples of buffering agents include, but are not limited to, 2-Amino-2-methyl-1,3-propanediol, ≧99.5% (NT), 2-Amino-2-methyl-1-propanol, ≧99.0% (GC), L-(+)-Tartaric acid, ≧99.5% (T), ACES, ≧99.5% (T), ADA, ≧99.0% (T), Acetic acid, ≧99.5% (GC/T), Acetic acid, for luminescence, ≧99.5% (GC/T), Ammonium acetate solution, for molecular biology, ˜5 M in H₂O, Ammonium acetate, for luminescence, ≧99.0% (calc. on dry substance, T), Ammonium bicarbonate, ≧99.5% (T), Ammonium citrate dibasic, ≧99.0% (T), Ammonium formate solution, 10 M in H₂O, Ammonium formate, ≧99.0% (calc. based on dry substance, NT), Ammonium oxalate monohydrate, ≧99.5% (RT), Ammonium phosphate dibasic solution, 2.5 M in H₂O, Ammonium phosphate dibasic, ≧99.0% (T), Ammonium phosphate monobasic solution, 2.5 M in H₂O, Ammonium phosphate monobasic, ≧99.5% (T), Ammonium sodium phosphate dibasic tetrahydrate, ≧99.5% (NT), Ammonium sulfate solution, for molecular biology, 3.2 M in H₂O, Ammonium tartrate dibasic solution, 2 M in H₂O (colorless solution at 20° C.), Ammonium tartrate dibasic, ≧99.5% (T), BES buffered saline, for molecular biology, 2× concentrate, BES, ≧99.5% (T), BES, for molecular biology, ≧99.5% (T), BICINE buffer Solution, for molecular biology, 1 M in H₂O, BICINE, ≧99.5% (T), BIS-TRIS, ≧99.0% (NT), Bicarbonate buffer solution, >0.1 M Na₂CO₃, >0.2 M NaHCO₃, Boric acid, ≧99.5% (T), Boric acid, for molecular biology, ≧99.5% (T), CAPS, ≧99.0% (TLC), CHES, ≧99.5% (T), Calcium acetate hydrate, ≧99.0% (calc. on dried material, KT), Calcium carbonate, precipitated, ≧99.0% (KT), Calcium citrate tribasic tetrahydrate, ≧98.0% (calc. on dry substance, KT), Citrate Concentrated Solution, for molecular biology, 1 M in H₂O, Citric acid, anhydrous, ≧99.5% (T), Citric acid, for luminescence, anhydrous, ≧99.5% (T), Diethanolamine, ≧99.5% (GC), EPPS, ≧99.0% (T), Ethylenediaminetetraacetic acid disodium salt dihydrate, for molecular biology, ≧99.0% (T), Formic acid solution, 1.0 M in H₂O, Gly-Gly-Gly, ≧99.0% (NT), Gly-Gly, ≧99.5% (NT), Glycine, ≧99.0% (NT), Glycine, for luminescence, ≧99.0% (NT), Glycine, for molecular biology, ≧99.0% (NT), HEPES buffered saline, for molecular biology, 2× concentrate, HEPES, ≧99.5% (T), HEPES, for molecular biology, ≧99.5% (T), Imidazole buffer Solution, 1 M in H₂O, Imidazole, ≧99.5% (GC), Imidazole, for luminescence, ≧99.5% (GC), Imidazole, for molecular biology, ≧99.5% (GC), Lipoprotein Refolding Buffer, Lithium acetate dihydrate, ≧99.0% (NT), Lithium citrate tribasic tetrahydrate, ≧99.5% (NT), MES hydrate, ≧99.5% (T), MES monohydrate, for luminescence, ≧99.5% (T), MES solution, for molecular biology, 0.5 M in H₂O, MOPS, ≧99.5% (T), MOPS, for luminescence, ≧99.5% (T), MOPS, for molecular biology, ≧99.5% (T), Magnesium acetate solution, for molecular biology, ˜1 M in H₂O, Magnesium acetate tetrahydrate, ≧99.0% (KT), Magnesium citrate tribasic nonahydrate, ≧98.0% (calc. based on dry substance, KT), Magnesium formate solution, 0.5 M in H₂O, Magnesium phosphate dibasic trihydrate, ≧98.0% (KT), Neutralization solution for the in-situ hybridization for in-situ hybridization, for molecular biology, Oxalic acid dihydrate, ≧99.5% (RT), PIPES, ≧99.5% (T), PIPES, for molecular biology, ≧99.5% (T), Phosphate buffered saline, solution (autoclaved), Phosphate buffered saline, washing buffer for peroxidase conjugates in Western Blotting, 10× concentrate, piperazine, anhydrous, ≧99.0% (T), Potassium D-tartrate monobasic, ≧99.0% (T), Potassium acetate solution, for molecular biology, Potassium acetate solution, for molecular biology, 5 M in H₂O, Potassium acetate solution, for molecular biology, ˜1 M in H₂O, Potassium acetate, ≧99.0% (NT), Potassium acetate, for luminescence, ≧99.0% (NT), Potassium acetate, for molecular biology, ≧99.0% (NT), Potassium bicarbonate, ≧99.5% (T), Potassium carbonate, anhydrous, ≧99.0% (T), Potassium chloride, ≧99.5% (AT), Potassium citrate monobasic, ≧99.0% (dried material, NT), Potassium citrate tribasic solution, 1 M in H₂O, Potassium formate solution, 14 M in H₂O, Potassium formate, ≧99.5% (NT), Potassium oxalate monohydrate, ≧99.0% (RT), Potassium phosphate dibasic, anhydrous, ≧99.0% (T), Potassium phosphate dibasic, for luminescence, anhydrous, ≧99.0% (T), Potassium phosphate dibasic, for molecular biology, anhydrous, ≧99.0% (T), Potassium phosphate monobasic, anhydrous, ≧99.5% (T), Potassium phosphate monobasic, for molecular biology, anhydrous, ≧99.5% (T), Potassium phosphate tribasic monohydrate, ≧95% (T), Potassium phthalate monobasic, ≧99.5% (T), Potassium sodium tartrate solution, 1.5 M in H₂O, Potassium sodium tartrate tetrahydrate, ≧99.5% (NT), Potassium tetraborate tetrahydrate, ≧99.0% (T), Potassium tetraoxalate dihydrate, ≧99.5% (RT), Propionic acid solution, 1.0 M in H₂O, STE buffer solution, for molecular biology, pH 7.8, STET buffer solution, for molecular biology, pH 8.0, Sodium 5,5-diethylbarbiturate, ≧99.5% (NT), Sodium acetate solution, for molecular biology, ˜3 M in H₂O, Sodium acetate trihydrate, ≧99.5% (NT), Sodium acetate, anhydrous, ≧99.0% (NT), Sodium acetate, for luminescence, anhydrous, ≧99.0% (NT), Sodium acetate, for molecular biology, anhydrous, ≧99.0% (NT), Sodium bicarbonate, ≧99.5% (T), Sodium bitartrate monohydrate, ≧99.0% (T), Sodium carbonate decahydrate, ≧99.5% (T), Sodium carbonate, anhydrous, ≧99.5% (calc. on dry substance, T), Sodium citrate monobasic, anhydrous, ≧99.5% (T), Sodium citrate tribasic dihydrate, ≧99.0% (NT), Sodium citrate tribasic dihydrate, for luminescence, ≧99.0% (NT), Sodium citrate tribasic dihydrate, for molecular biology, ≧99.5% (NT), Sodium formate solution, 8 M in H₂O, Sodium oxalate, ≧99.5% (RT), Sodium phosphate dibasic dihydrate, ≧99.0% (T), Sodium phosphate dibasic dihydrate, for luminescence, ≧99.0% (T), Sodium phosphate dibasic dihydrate, for molecular biology, ≧99.0% (T), Sodium phosphate dibasic dodecahydrate, ≧99.0% (T), Sodium phosphate dibasic solution, 0.5 M in H₂O, Sodium phosphate dibasic, anhydrous, ≧99.5% (T), Sodium phosphate dibasic, for molecular biology, ≧99.5% (T), Sodium phosphate monobasic dihydrate, ≧99.0% (T), Sodium phosphate monobasic dihydrate, for molecular biology, ≧99.0% (T), Sodium phosphate monobasic monohydrate, for molecular biology, ≧99.5% (T), Sodium phosphate monobasic solution, 5 M in H₂O, Sodium pyrophosphate dibasic, ≧99.0% (T), Sodium pyrophosphate tetrabasic decahydrate, ≧99.5% (T), Sodium tartrate dibasic dihydrate, ≧99.0% (NT), Sodium tartrate dibasic solution, 1.5 M in H₂O (colorless solution at 20° C.), Sodium tetraborate decahydrate, ≧99.5% (T), TAPS, ≧99.5% (T), TES, ≧99.5% (calc. based on dry substance, T), TM buffer solution, for molecular biology, pH 7.4, TNT buffer solution, for molecular biology, pH 8.0, TRIS Glycine buffer solution, 10× concentrate, TRIS acetate-EDTA buffer solution, for molecular biology, TRIS buffered saline, 10× concentrate, TRIS glycine SDS buffer solution, for electrophoresis, 10× concentrate, TRIS phosphate-EDTA buffer solution, for molecular biology, concentrate, 10× concentrate, Tricine, ≧99.5% (NT), Triethanolamine, ≧99.5% (GC), Triethylamine, ≧99.5% (GC), Triethylammonium acetate buffer, volatile buffer, ˜1.0 M in H₂O, Triethylammonium phosphate solution, volatile buffer, ˜1.0 M in H₂O, Trimethylammonium acetate solution, volatile buffer, ˜1.0 M in H₂O, Trimethylammonium phosphate solution, volatile buffer, ˜1 M in H₂O, Tris-EDTA buffer solution, for molecular biology, concentrate, 100× concentrate, Tris-EDTA buffer solution, for molecular biology, pH 7.4, Tris-EDTA buffer solution, for molecular biology, pH 8.0, Trizma® acetate, ≧99.0% (NT), Trizma® base, ≧99.8% (T), Trizma® base, ≧99.8% (T), Trizma® base, for luminescence, ≧99.8% (T), Trizma® base, for molecular biology, ≧99.8% (T), Trizma® carbonate, ≧98.5% (T), Trizma® hydrochloride buffer solution, for molecular biology, pH 7.2, Trizma® hydrochloride buffer solution, for molecular biology, pH 7.4, Trizma® hydrochloride buffer solution, for molecular biology, pH 7.6, Trizma® hydrochloride buffer solution, for molecular biology, pH 8.0, Trizma® hydrochloride, ≧99.0% (AT), Trizma® hydrochloride, for luminescence, ≧99.0% (AT), Trizma® hydrochloride, for molecular biology, ≧99.0% (AT), and Trizma® maleate, ≧99.5% (NT).

The nanoemulsion can comprise one or more emulsifying agents to aid in the formation of emulsions. Emulsifying agents include compounds that aggregate at the oil/water interface to form a kind of continuous membrane that prevents direct contact between two adjacent droplets. Certain embodiments of the present invention feature nanoemulsions that may readily be diluted with water to a desired concentration without impairing their anti-fungal or antiyeast properties.

### D. PHARMACEUTICAL COMPOSITIONS

The nanoemulsions of the invention may be formulated into pharmaceutical compositions that comprise the nanoemulsion in a therapeutically effective amount and suitable, pharmaceutically-acceptable excipients for topical administration to a human subject in need thereof. Such excipients are well known in the art.

By the phrase “therapeutically effective amount” it is meant any amount of the nanoemulsion that is effective in preventing and/or treating acne. One possible way to treat acne is by killing or inhibiting the growth of P. acnes, causing P. acnes to lose pathogenicity, or any combination thereof.

Topical administration includes administration to the skin, including surface of the hair follicle and pilosebaceous unit.

Pharmaceutically acceptable dosage forms for topical administration include, but are not limited to, ointments, creams, liquids, emulsions, lotions, gels, bioadhesive gels, aerosols, pastes, foams, sunscreens, or in the form of an article or carrier, such as a bandage, insert, syringe-like applicator, pessary, powder, talc or other solid, cleanser (leave on and wash off product), and agents that favor penetration within the pilosebaceous gland.

The pharmaceutical compositions may be formulated for immediate release, sustained release, controlled release, delayed release, or any combinations thereof, into the epidermis or dermis, with no systemic absorption. In some embodiments, the formulations may comprise a penetration-enhancing agent for enhancing penetration of the nanoemulsion through the stratum corneum and into the epidermis or dermis. Suitable penetration-enhancing agents include, but are not limited to, alcohols such as ethanol, triglycerides and aloe compositions. The amount of the penetration-enhancing agent may comprise from about 0.5% to about 40% by weight of the formulation.

In some embodiments, the formulation for delivery via a “patch” comprising a therapeutically effective amount of the nanoemulsion is envisioned. As used herein a “patch” comprises at least a topical formulation and a covering layer, such that the patch can be placed over the area to be treated. Preferably, the patch is designed to maximize delivery through the stratum corneum and into the epidermis or dermis, while minimizing absorption into the circulatory system, and little to no skin irritation, reducing lag time, promoting uniform absorption, and reducing mechanical rub-off and dehydration.

Adhesives for use with the drug-in-adhesive type patches are well known in the art. Suitable adhesive include, but are not limited to, polyisobutylenes, silicones, and acrylics. These adhesives can function under a wide range of conditions, such as, high and low humidity, bathing, sweating etc. Preferably the adhesive is a composition based on natural or synthetic rubber; a polyacrylate such as, polybutylacrylate, polymethylacrylate, poly-2-ethylhexyl acrylate; polyvinylacetate; polydimethylsiloxane; or and hydrogels (e.g., high molecular weight polyvinylpyrrolidone and oligomeric polyethylene oxide). The most preferred adhesive is a pressure sensitive acrylic adhesive, for example Durotak® adhesives (e.g., Durotak® 2052, National Starch and Chemicals). The adhesive may contain a thickener, such as a silica thickener (e.g., Aerosil, Degussa, Ridgefield Park, N.J.) or a crosslinker such as aluminumacetylacetonate.

Suitable release liners include but are not limited to occlusive, opaque, or clear polyester films with a thin coating of pressure sensitive release liner (e.g., silicone-fluorsilicone, and perfluorcarbon based polymers.

Backing films may be occlusive or permeable and are derived from synthetic polymers like polyolefin oils polyester, polyethylene, polyvinylidine chloride, and polyurethane or from natural materials like cotton, wool, etc. Occlusive backing films, such as synthetic polyesters, result in hydration of the outer layers of the stratum corneum while non-occlusive backings allow the area to breath (i.e., promote water vapor transmission from the skin surface). More preferably the backing film is an occlusive polyolefin foil (Alevo, Dreieich, Germany). The polyolefin foil is preferably about 0.6 to about 1 mm thick.

The shape of the patch can be flat or three-dimensional, round, oval, square, and have concave or convex outer shapes, or the patch or bandage can also be segmented by the user into corresponding shapes with or without additional auxiliary means.

The nanoemulsions of the invention can be applied and/or delivered utilizing electrophoretic delivery/electrophoresis. Such transdermal methods, which comprise applying an electrical current, are well known in the art.

The pharmaceutical compositions for topical administration may be applied in a single administration or in multiple administrations. The pharmaceutical compositions are topically applied for at least once a week, at least twice a week, at least once a day, at least twice a day, multiple times daily, multiple times weekly, biweekly, at least once a month, or any combination thereof. The pharmaceutical compositions are topically applied for a period of time of about one month, about two months, about three months, about four months, about five months, about six months, about seven months, about eight months, about nine months, about ten months, about eleven months, about one year, about 1.5 years, about 2 years, about 2.5 years, about 3 years, about 3.5 years, about 4 years, about 4.5 years, and about 5 years. Between applications, the application area may be washed to remove any residual nanoemulsion.

Preferably, the pharmaceutical compositions are applied to the skin area in an amount of from about 0.001 mL/cm² to about 5.0 mL/cm². An exemplary application amount and area is about 0.2 mL/cm², although any amount from 0.001 mL/cm² up to about 5.0 mL/cm² can be applied. Following topical administration, the nanoemulsion may be occluded or semi-occluded. Occlusion or semi-occlusion may be performed by overlaying a bandage, polyoleofin film, impermeable barrier, or semi-impermeable barrier to the topical preparation. Preferably, after application, the treated area is covered with a dressing.

### E. EXEMPLARY NANOEMULSIONS

Several exemplary nanoemulsions are described below, although the methods of the invention are not limited to the use of such nanoemulsions. The components and quantity of each can be varied as described herein in the preparation of other nanoemulsions. Unless otherwise noted, all concentrations are expressed in terms of % w/w.

TABLE 1
Exemplary Therapeutically Effective Nanoemulsions

| | Soybean | Tween 20 | | CPC % | EDTA | H₂O |
|---|---|---|---|---|---|---|
| Form. (CPC % w/v) | oil % | % | Ethanol % | (mg/mL) | % (mM) | % |
| Formulation #1; 0.50% | 31.4 | 2.96 | 3.37 | 0.53 (5) | 0.037 (1) | 61.70 |
| Formulation #2; 0.25% | 15.7 | 1.48 | 1.68 | 0.27 (2.5) | 0.0185 (0.5) | 80.85 |
| Formulation #3; 1.0% | 62.79 | 5.92 | 6.73 | 1.068 (10) | 0.075 (2) | 23.42 |
| Formulation #4; 0.3% | 18.84 | 1.78 | 2.02 | 0.320 (3) | 0.0224 (0.6) | 77.03 |
| Formulation #5; 0.1% | 6.28 | 0.59 | 0.67 | 0.107 (1) | 0.0075 (0.2) | 92.34 |

Several additional exemplary nanoemulsions are described below. For therapeutic topical use on a subject, the concentrations of each component would be increased, as described above.

TABLE 2
Exemplary Nanoemulsions

| Form. | Soybean | Tween 20 | | CPC % | EDTA | H2O |
|---|---|---|---|---|---|---|
| (CPCw/v %) | oil % | % | Ethanol % | (μg/mL) | % (uM) | % |
| Formulation | 0.050 | 0.00474 | 0.00538 | 0.00085 (8) | 5.96 × 10⁻⁵ (1.6) | 99.94 |
| #6; 0.0008% | | | | | | |
| Formulation | 0.025 | 0.00237 | 0.00269 | 0.00043 (4) | 2.98 × 10^{-5 (}0.8) | 99.97 |
| #7; 0.0004% | | | | | | |
| Formulation | 0.013 | 0.00118 | 0.00135 | 0.00021 (2) | 1.49 × 10⁻⁵ (0.4) | 99.98 |
| #8; 0.0002% | | | | | | |

### F. METHODS OF MANUFACTURE

The nanoemulsions of the invention can be formed using classic emulsion forming techniques. See e.g., U.S. 2004/0043041. See also the method of manufacturing nanoemulsions described in U.S. Pat. Nos. 6,559,189, 6,506,803, 6,635,676, 6,015,832, and U.S. Patent Publication Nos. 20040043041, 20050208083, 20060251684, and 20070036831, and WO 05/030172, all of which are specifically incorporated by reference. In an exemplary method, the oil is mixed with the aqueous phase under relatively high shear forces (e.g., using high hydraulic and mechanical forces) to obtain a nanoemulsion comprising oil droplets having an average diameter of less than about 1000 nm. Some embodiments of the invention employ a nanoemulsion having an oil phase comprising an alcohol such as ethanol. The oil and aqueous phases can be blended using any apparatus capable of producing shear forces sufficient to form an emulsion, such as French Presses or high shear mixers (e.g., FDA approved high shear mixers are available, for example, from Admix, Inc., Manchester, N.H.). Methods of producing such emulsions are described in U.S. Pat. Nos. 5,103,497 and 4,895,452, herein incorporated by reference in their entireties.

In an exemplary embodiment, the nanoemulsions used in the methods of the invention comprise droplets of an oily discontinuous phase dispersed in an aqueous continuous phase, such as water. The nanoemulsions of the invention are stable, and do not decompose even after long storage periods. Certain nanoemulsions of the invention are non-toxic and safe when swallowed, inhaled, or contacted to the skin of a subject.

The compositions of the invention can be produced in large quantities and are stable for many months at a broad range of temperatures. The nanoemulsion can have textures/consistencies ranging from that of a semi-solid cream to that of a thin lotion and can be applied topically by hand and sprayed onto a surface. As stated above, at least a portion of the emulsion may be in the form of lipid structures including, but not limited to, unilamellar, multilamellar, and paucliamellar lipid vesicles, micelles, and lamellar phases.

The present invention contemplates that many variations of the described nanoemulsions will be useful in the methods of the present invention. To determine if a candidate nanoemulsion is suitable for use with the present invention, three criteria are analyzed. Using the methods and standards described herein, candidate emulsions can be easily tested to determine if they are suitable. First, the desired ingredients are prepared using the methods described herein, to determine if a nanoemulsion can be formed. If a nanoemulsion cannot be formed, the candidate is rejected. Second, the candidate nanoemulsion should form a stable emulsion. A nanoemulsion is stable if it remains in an emulsion form for a sufficient period to allow its intended use. For example, for nanoemulsions that are to be stored, shipped, etc., it may be desired that the nanoemulsion remain in emulsion form for months to years. Typical nanoemulsions that are relatively unstable, will lose their form within a day. Third, the candidate nanoemulsion should have efficacy for its intended use. For example, the emulsions of the invention should kill or disable Propionibacterium species in vitro or reduce inflammation and/or non-inflammatory lesions in humans. To determine the potency of a particular candidate nanoemulsion against P. acnes, MICs are determined under standardized conditions (National Committee for Clinical Laboratory Standards, Methods for Antimicrobial Susceptibility Testing of Anaerobic Bacteria, 7^{th} ed.;” Approved Standard M11-A7. National Committee for Clinical Laboratory Standards, Wayne, Pa. (2007)).

Alternatively, P. acnes can be exposed to the nanoemulsion for one or more time periods in a side-by-side experiment with an appropriate control sample (e.g., a negative control such as water) and determining if, and to what degree, the nanoemulsion kills or disables P. acnes.

The nanoemulsion of the invention can be provided in many different types of containers and delivery systems. For example, in some embodiments of the invention, the nanoemulsions are provided in a cream or other solid or semi-solid form. The nanoemulsions of the invention may be incorporated into hydrogel formulations.

The nanoemulsions can be delivered (e.g., to a subject or customers) in any suitable container. Suitable containers can be used that provide one or more single use or multi-use dosages of the nanoemulsion for the desired application. In some embodiments of the invention, the nanoemulsions are provided in a suspension or liquid form. Such nanoemulsions can be delivered in any suitable container including spray bottles (e.g., pressurized spray bottles).

### G. EXAMPLES

The invention is further described by reference to the following examples, which are provided for illustration only. The invention is not limited to the examples, but rather includes all variations that are evident from the teachings provided herein. All publicly available documents referenced herein, including but not limited to U.S. patents, are specifically incorporated by reference.

### Example 1

### Preparation of Nanoemulsions

These emulsions are produced by mixing a water-immiscible oil phase into an aqueous phase with a proprietary manufacturing method. The two phases (aqueous phase and oil phase) are combined together and processed to yield an emulsion. The emulsion is further processed to achieve the desired particle size. For the gel formulation, a thickening agent, such as Klucel can be added to the nanoemulsion. For example, Klucel is dissolved in water or any aqueous solvent and added to the nanoemulsion to achieve the desired concentration.

### Example 2

### The Nanoemulsions have Potent Activity Against P. acnes

Nanoemulsions according to the invention were tested in in vitro to determine the minimum inhibitory concentration (MIC) and minimum bactericidal concentration (MBC) against 16 clinical isolates of P. acnes, some of which have defined ribosomally-based resistance mechanisms to erythromycin, clindamycin and/or tetracycline. The nanoemulsions (“NB-00X”) comprised, in an aqueous medium, soybean oil, Tween 20® as a nonionic surfactant, ethanol, cetylpyridinium chloride (CPC) as a cationic surfactant, EDTA, and water, and optionally, a thickening agent for the gel formulation.

TABLE 3
Compositions of the Nanoemulsions (NB-00X) and Nanoemulsion Gels (NB-Gel).
The percentages are wt/wt, unless otherwise noted.

| | Lot | Soybean | Tween 20 | Ethanol | CPC | EDTA | Klucel | Water |
|---|---|---|---|---|---|---|---|---|
| Formulation | # | oil % | % | % | % (w/v) | % | % | % |
| 0.1% NB-00X | 89-16-09A | 6.279 | 0.592 | 0.679 | 0.107 | 0.0074 | 0 | 92.34 |
| 0.3% NB-00X | X-1160 | 18.837 | 1.776 | 2.037 | 0.320 | 0.022 | 0 | 77.01 |
| 0.1% NB-Gel | 89-16-09C | 6.279 | 0.592 | 20.679 | 0.107 | 0.0074 | 1% | 92.34 |
| 0.3% NB-Gel | 89-7025 | 18.837 | 1.776 | 22.037 | 0.320 | 0.022 | 1% | 77.01 |

The nanoemulsions were tested at 10 different concentrations, as two-fold serial dilutions from 0.0064% NB-00X (equivalent to 64 μg CPC/ml) to 0.0000125% NB-00X (equivalent to 0.125 μg CPC/ml). Each dilution contained varying concentrations of soybean oil, Tween 20®, ethanol, CPC, and EDTA. Combination products were also evaluated; stock emulsions containing NB-00X gel (3 mg CPC/ml)+2% salicyclic acid or NB-00X gel+0.5% benzoyl peroxide (BPO) were serially diluted two-fold and each concentration was tested against 16 P. acnes isolates. In general, the standard methodology was followed for MIC and MBC determination.

The MIC (minimum inhibitor concentration) and MBC (minimum bactericidal concentration) values for the nanoemulsions were compared to the MIC and MBC values of anti-acne drugs currently in use: erythromycin, clindamycin, tetracycline, benzoyl peroxide and salicylic acid.

A. Source of Drugs and P. acnes Isolates

NB-00X (liquid formulation), lot X1151 and NB-00X gel, lot X1158, were prepared at concentrations of 6000 μg/ml and 3000 μg/ml respectively. These lots were prepared at NanoBio Corporation from NB-PO-004-FP manufactured at Contract Pharmaceutical Laboratories (CPL), Buffalo, N.Y., USA. Placebo lots X1161 and X1162 (placebo for NB-00X gel, contains thickening agent and additional solvent) were prepared from lot A0494 manufactured at NanoBio Corporation. Since nanoemulsions are not a single small molecule, their relative activity can be expressed in terms of the concentration of cationic surfactant present. Thus, the antibacterial activity of NB-00X formulations is expressed in microgram CPC per ml. NB-00X gel (lot X1158) contained a thickening agent in addition to the components of NB-00X. Combination products were made as stock emulsions containing NB-00X gel (3 mg CPC/ml)+2% salicyclic acid or NB-00X gel+0.5% benzoyl peroxide (BPO).

Comparator compounds, erythromycin, clindamycin, tetracycline and chlorhexidine were purchased from Sigma Chemicals, USP, Fluka and Aldrich as catalog numbers E0774, 1136002, 87128, and 282227 respectively. Salicylic acid was purchased from J. T. Baker as VWR International catalog number 0300-01. BPO in the form of Invisible Acne cream containing 10% BPO was purchased from Meijer Distribution Inc. (Grand Rapids, Mich.).

The source of bacterial strains was mainly Basilea Pharmaceutica, AG, Basel, Switzerland (Heller, S., L. Kellenberger and S. Shapiro, 2007, Antipropionibacterial activity of BAL19403, A Novel Macrolide Antibiotic, J. Antimicrob. Chemother. 51: 1956-1961). The majority of these isolates had defined resistance mechanisms to erythromycin, clindamycin and/or tetracycline. The resistance mechanisms were mutations in either the 16S or 23S rRNA of the small or large ribosomal subunit conferring tetracycline or erythromycin±clindamycin resistance, respectively, or resistance was conferred by an erm(X) methylase that dimethylates residue A2058 in 23S rRNA, conferring high level erythromycin and clindamycin resistance. Three isolates were obtained from the American Type Culture Collection (ATCC), Manassas, Va., USA.

B. Preparation of Drug Concentrations

Weighing of drugs and potency calculations were done as prescribed by Clinical and Laboratory Standards Institute (1). Dimethyl sulfoxide (DMSO) was used to prepare stock solutions of the water-insoluble compound tetracycline at 100× concentrations. Stock solutions of erythromycin and clindamycin were prepared in sterile deionized water (DI water) at a 100× of the highest test concentration. Stock solutions of chlorhexidine and NB-00X were prepared at a 4× concentration in DI water.

C. Preparation of 96 Well Drug Plates

To prepare intermediate concentrations, 100× stock solutions were serially diluted 1:1 using DMSO or DI water. Final concentrations were made by 1:50 or 1:1 dilutions in Wilkin Chalgren media (1) to give 2× of the test concentrations, with final DMSO concentrations at 1%. 50 μl of these drug concentrations were transferred to 96-well plates using multi-channel pipettes.

D. Determination of MIC and MBC

P. acnes strains grown on sheep blood agar for 24-48 hrs at 35° C. were used as the sources of inocula for susceptibility studies as per Clinical and Laboratory Standards Institute. A bacterial suspension with turbidity equivalent to a 0.5 McFarland standard was diluted to 1:75 in saline or Wilkins-Chalgren broth (GLP Corporation) to give >10⁶ cfu/ml in each well after inoculation. Within 15 minutes, each well (except the negative growth controls) of the microtiter tray containing the serial dilutions of test compounds received 50 μl of inoculum, resulting into a log₂ dilution of both drug and bug in each well. Verification of the colony-forming units in the inoculum was performed by diluting the adjusted inoculum preparation to 10⁴ and plating 100 μl on blood agar plate.

Microtiter and blood agar plates were incubated at 35-37° C. for 48 h in a 7.0 L AnaeroPack Jar (Mitsubishi gas chemical; No. 50-70) fitted with an anaerobic gas generating system (Misubishi, No. 10-01) and a dry anaerobic indicator strip (BBL, Becton, Dickinson & Co. #271051). MICs were read visually using a 96-well plate reader fitted with a magnifying mirror (Biodesign of New York). Because of the opacity of benzoyl peroxide, 20 μl of Cell Titer Blue (alamar blue from Promega G8080) was added after 48 hrs; the plates were incubated for an additional hour prior to reading. Colony-forming units were counted after 72 h of incubation to ensure that the initial inocula were between 2-5×10⁶ cfu/ml.

The minimal bactericidal concentrations (MBC) for P. acnes were determined by plating 10 μl from the well determined to be the MIC plus 4 wells above the MIC on blood-supplemented Mueller-Hinton agar plate. Inoculated petri plates were incubated for 72 h at 35° C. under anaerobic conditions. The MBC was calculated as the concentration of drug that gave ≧3-log reduction from the initial inoculum concentration.

MICs for NB-00X or NB-00X gel (formulation modified to include a thickening agent and additional solvent) ranged from 0.25-1.0 μg/ml and MBCs ranged from 0.5-4 μg/ml (Table 4). The MIC₉₀ and MBC₉₀ values were 0.5 μg/ml and 2.0 μg/ml for NB-00X and 1 μg/ml and 2 μg/ml for NB-00X gel, respectively. Greater than 50% of the isolates were resistant to erythromycin and clindamycin; 44% of the isolates were resistant to tetracycline. However, multidrug-resistant isolates were equally susceptible to either formulation of NB-00X. Neither placebo had any microbiological activity. NB-00X was bactericidal against all the isolates, including strains that were erythromycin-, clindamycin- and/or tetracycline-resistant. The MICs and MBCs of chlorhexidine for all the strains were at or below the lowest tested level of 10 μg/ml (equivalent to 0.001% chlorhexidine).

Since NB-00X is a nanoemulsion and is preferentially taken up by the transfollicular route (Ciotti et al., “Novel nanoemulsion NB-001 permeates skin by the follicular route,” Abstr. 45^{th} Intersci. Conf., Antimicrob. Agents Chemother., abstr. A-1898 (2008)), incorporation of another anti-acne drug into the nanodroplets could be used to effectively deliver these additional agents to the site of infection. Thus, we looked at the microbiological activity of NB-00X gel formulated with either benzoyl peroxide or salicyclic acid and compared the MICs and MBCs of the combination products to benzoyl peroxide or salicyclic acid alone. Since neither BPO or salicyclic acid were highly potent (MIC₉₀ values of 50 and 1000 μg/ml, respectively), the antimicrobial activity seen with the combination products NB-00X+BPO or NB-00X+salicyclic acid reflected the intrinsic activity of NB-00X, with MIC₉₀ values of 0.5 μg/ml for either combination and MBC₉₀ values of 4 and 2 μg/ml, respectively.

NB-00X has relevant microbiological and bactericidal activity against a collection of recent clinical isolates of P. acnes, including multidrug-resistant strains. Comparator drugs that have been used to treat acne—erythromycin, clindamycin, tetracycline, benzoyl peroxide and salicyclic acid—were much less effective. Combinations of the nanoemulsion NB-00X with BPO or salicyclic acid were as effective as NB-00X alone. However, given the transfollicular route of NB-00X (2), additional acne agents could be delivered more effectively to the site of infection.

TABLE 4
MIC₉₀/MBC₉₀ Values Against 16 P. acnes Isolates

| | | | MIC₅₀/MBC₅₀ | | MIC₉₀/MBC₉₀ | |
|---|---|---|---|---|---|---|
| | Range | | Value | | Value | |
| Compound | MIC | MBC | MIC₅₀ | MBC₅₀ | MIC₉₀ | MBC₉₀ |
| NB-00X | 0.25-1 | 0.5-2 | 0.5 | 2 | 0.5 | 2 |
| NB-00X gel | 0.5-1 | 1-4 | 0.5 | 2 | 1 | 2 |
| Erythromycin | ≦0.25->128 | >4->128 | 2 | >64 | >128 | >128 |
| Clindamycin | ≦0.125->64 | >2->64 | 2 | >64 | >64 | >64 |
| Tetracycline | ≦0.063->32 | >1->32 | 1 | >16 | >32 | >32 |
| Chlorhexidine | ≦10 | ≦10 | ≦10 | ≦10 | ≦10 | ≦10 |
| Benzoyl peroxide | ≦50-100 | 100-400 | 50 | 200 | 50 | 200 |
| Salicylic acid | 500-1000 | 2000->2000 | 1000 | 2000 | 1000 | 2000 |
| NB-00X placebo | >64 | >64 | >64 | >64 | >64 | >64 |
| NB-00X gel | >64 | >64 | >64 | >64 | >64 | >64 |
| placebo | | | | | | |
| NB-00X + BPO^{a} | 0.5-1 | 2-4 | 0.5 | 4 | 0.5 | 4 |
| NB-00X + SA^{b} | 0.25-1 | 0.5-2 | 0.5 | 2 | 0.5 | 2 |

^{a}NB-00X gel (3 mg CPC/ml) + 0.5% BPO
^{b}NB-00X gel (3 mg CPC/ml) + 2% salicyclic acid

### Example 3

### The Nanoemulsions have Potent Activity Against P. acnes in the Presence of Sebum

Nanoemulsions according to the invention were tested in in vitro antibacterial assays in the presence of 50% artificial sebum to determine the minimum inhibitory concentration (MIC) and minimum bactericidal concentration (MBC) against 16 clinical isolates of P. acnes. The nanoemulsions (“NB-002”) comprised, in an aqueous medium, soybean oil, Tween 20® as a nonionic surfactant, ethanol, cetylpyridinium chloride (CPC) as a cationic surfactant, EDTA, and water.

TABLE 5
Composition of NB-00X formulations

| | | Soybean | Tween 20 | | CPC % | EDTA | Klucel | Water |
|---|---|---|---|---|---|---|---|---|
| Formulation | Lot # | oil % | % | Ethanol % | (w/v) | % | % | % |
| 0.1% NB-00X | 89-16-09A | 6.279 | 0.592 | 0.679 | 0.107 | 0.0074 | 0 | 92.34 |
| 0.3% NB-00X | X-1160 | 18.837 | 1.776 | 2.037 | 0.320 | 0.022 | 0 | 77.01 |
| 0.1% NB-Gel | 89-16-09C | 6.279 | 0.592 | 20.679 | 0.107 | 0.0074 | 1% | 92.34 |
| 0.3% NB-Gel | 89-7025 | 18.837 | 1.776 | 22.037 | 0.320 | 0.022 | 1% | 77.01 |

The source of drugs and isolates were the same as in Example 2. 100X drug stocks were prepared as in Example 2.

A. Preparation of Artificial Sebum

Artificial sebum was prepared by adding the entire ingredients given in the Table 6 and heating at 60° C. in a water bath, with intermittent stirring until all solids melted resulting in to a clear yellow liquid (Valiveti et al., “Diffusion properties of model compounds in artificial sebum. Int. J. Pharm., 345:88-94 (2007)).

TABLE 6
Composition of artificial sebum.

| | | | | Amt |
|---|---|---|---|---|
| | | Lot | w/w | (g) for |
| Ingredient | Manufacturer | # | % | 200 g |
| Oleic acid | Aldrich | 10529CH | 1.4 | 2.80 |
| Palmitoleic acid | MP Biomedical | 8855J | 5 | 10.00 |
| Squalene | MP Biomedical | 7501F | 15 | 30.00 |
| Olive oil | Spectrum | XA0813 | 10 | 50.00 |
| (C16-18) | | | | |
| Cottonseed oil | Spectrum | XC1142 | 25 | 50.00 |
| (C16-18) | | | | |
| Cholesterol | JT Baker | E33H12 | 1.2 | 2.40 |
| Cholesterol oleate | Gantaur (Cat#21130) | 12373 | 2.4 | 4.80 |
| Palmitic acid | Calbiochem | D00013930 | 5 | 10.00 |
| Spermaceti wax | Aqua Solutions | 304601 | 15 | 30.00 |
| Paraffin wax | Sigma | 06007DE | 10 | 20.00 |
| (mp 58-62 C.) | | | | |
| Coconut oil | Aldrich | A0162449 | 10 | 20.00 |
| (C12-16) | | | | |

B. Preparation of Drug Plates

To prepare intermediate concentrations, 100× stock solutions were serially diluted 1:1 using DMSO or DI water. Final concentrations were made by 1:50 dilutions in Wilkin Chalgren media to give 2× of the final test concentrations, with final DMSO concentrations at 1%. 50 μl of these drug concentrations were transferred to 96-well plates using multi-channel pipettes.

Sebum was prewarmed to 50° C. and each well received 45 μl of sebum. After ten minutes at 35° C., five microliters of a P. acnes culture at 10⁸ colony-forming units/ml was added to each well. Plates were incubated and MICs and MBCs determined as described in Example 2.

C. Results

NB-00X was compared to NB-00X gel and the combinations of 0.3% NB-00X gel (3 mg CPC/ml)+0.5% benzoyl peroxide (BPO) or NB-00X gel+2% salicylic acid (SA). The MIC₉₀ and MBC₉₀ values for NB-00X formulations and comparators against sixteen isolates of P. acnes in the presence of 50% sebum are shown in Table 7. NB-00X was bactericidal for all strains of P. acnes with MIC₉₀/MBC₉₀ values of 0.5/2 μg/ml in the absence of sebum (Table 4). The MIC₉₀/MBC₉₀ values in the presence of 50% sebum increased to 128/1024 μg/ml (Table 7). A reduction in the MBC₉₀ for NB-00X occurred when BPO or SA was integrated into the formulation, resulting in a MIC₉₀/MBC₉₀ of 128/256 μg/ml in the presence of 50% sebum. The MIC₉₀/MBC₉₀ values of SA (1000/2000 μg/ml) were not significantly impacted by the presence of sebum, but the MIC₉₀/MBC₉₀ values of BPO increased eight-fold in the presence of sebum (400/1600 μg/ml) (Tables 4 and 7). The addition of sebum also did not impact the microbiological activities of erythromycin, clindamycin and tetracycline, at least up to the concentrations tested (Tables 4 and 7). The MIC₉₀ of chlorhexidine in the presence of sebum increased at least eight-fold in the presence of sebum and the MBCs increased at least 125-fold (Tables 4 and 7).

TABLE 7
Susceptiblity of 16 P. acnes isolates in the presence of 50%
artificial sebum

MIC Values
(μg/ml)

| Active Substance | MIC₉₀ | MBC₉₀ |
|---|---|---|
| Erythromycin | >128 | >128 |
| Clindamycin | >64 | >64 |
| Tetracycline | 32 | >32 |
| Chlorhexidine | 78 | 1250 |
| NB-00X | 128 | 1024 |
| NB-00X gel | 128 | 1024 |
| NB-00X/Benzoyl peroxide gel | 128 | 256 |
| NB-00X/Salicylic acid gel | 128 | 256 |
| Benzoyl peroxide | 400 | 1600 |
| Salicylic acid | 1000 | 2000 |

### Example 4

### Skin Permeation Studies

The purpose of this example was to evaluate the in vitro absorption into the epidermis and dermis of nanoemulsions according to the invention. Pig skin was used as an animal model.

A. In Vitro Skin Model

The in vitro skin model has proven to be a valuable tool for the study of percutaneous absorption of topically applied compounds. The model uses excised skin mounted in specially designed diffusion chambers that allow the skin to be maintained at a temperature and humidity that match typical in vivo conditions. (Franz, T J, “Percutaneous absorption: on the relevance of in vitro data,” J. Invest. Dermatol., 64:190-195 (1975).) A finite dose of formulation is applied to the epidermis, and outer surface of the skin and compound absorption is measured by monitoring its rate of appearance in the receptor solution bathing the dermal surface of the skin. Data defining total absorption, rate of absorption, as well as skin content can be accurately determined in this model. The method has historic precedent for accurately predicting in vivo percutaneous absorption kinetics. (Franz T J, “The finite dose technique as a valid in vitro model for the study of percutaneous absorption in man,” In: Skin: Drug Application and Evaluation of Environmental Hazards, Current Problems in Dermatology, vol. 7, Simon et al. (Eds) (Basel, Switzerland, S. Karger, 1978, pp 58-68.)

B. Nanoemulsions Used in the Study

TABLE 8
Composition of the Formulations

| | | Soybean | Tween 20 | | CPC % | EDTA | Klucel | Water |
|---|---|---|---|---|---|---|---|---|
| Formulation | Lot # | oil % | % | Ethanol % | (w/v) | % | % | % |
| 0.1% NB-001 | 89-16-09A | 6.279 | 0.592 | 0.679 | 0.107 | 0.0074 | 0 | 92.34 |
| 0.3% NB-001 | X-1160 | 18.837 | 1.776 | 2.037 | 0.320 | 0.022 | 0 | 77.01 |
| 0.1% NB-Gel | 89-16-09C | 6.279 | 0.592 | 20.679 | 0.107 | 0.0074 | 1% | 92.34 |
| 0.3% NB-Gel | 89-7025 | 18.837 | 1.776 | 22.037 | 0.320 | 0.022 | 1% | 77.01 |

C. Pig Skin

Full thickness, abdominal skin (˜1000 μm thickness) from 5.4 month old male Hanford swine (S/N 5353) was used in permeation studies and obtained from Sinclair Research Center, Inc, Auxvasse, Mo. The subcutaneous fat was removed using a scalpel and the skin was stored in aluminum foil pouches at −70° C. until use. At time of use, the skin was thawed by placing the sealed pouch in 30° C. water for approximately five minutes. Thawed skin was removed from the pouch and cut into circular discs (30 mm diameter) to fit between the donor and receiver sides of the permeation chambers.

D. Franz Diffusion Cell Methodology: Conditions, Parameters, Procedure

Percutaneous absorption was measured using the in vitro cadaver skin finite dose technique. Thirty mm of swine skin was placed onto the surface of each cell. Each receptor compartment was filled with distilled water, pH 7 and the donor compartment was left open to ambient laboratory conditions. The receptor compartment spout was covered with a screw cap to minimize evaporation of the receptor solution. All cells were mounted in a diffusion apparatus in which the receptor solution was maintained at 37° C. The receptor compartment was maintained at 34.5° C. in a water bath and was stirred with a magnetic stirrer.

The skin was equilibrated before applying 113 μL of each test article onto the skin surface.

E. Sampling (Receptor Sampling, Epidermis, Dermis, Surface Swabs)

Twenty-four hours after application of the first dose, the surface of the dosing area was rinsed with ethanol solution and swabbed independently to remove all residual formulation from the skin surface. Receptor solution was also sampled at 24 hours from the receptor of each cell and filtered into vials.

Skin samples were collected as described above; weights of the epidermal and dermal samples were obtained. The epidermal and dermal tissues were extracted with absolute ethanol, sonicated, and filtered and assayed using HPLC.

F. Epidermal and Dermal Calculations

The amount of CPC that permeated into the epidermis, dermis and the receptor compartment was determined by HPLC. A standard concentration of CPC was generated and used to determine the concentration of CPC in the dosing area. The levels of CPC in each skin area are represented as the amount per wet tissue weight (μg/grams)±the standard deviation.

The results of CPC permeation studies are shown in FIGS. 2 and 3. There was an increase in the delivery of the CPC marker to the epidermis and dermis with the 0.3% NB-00X as compared to the 0.1% NB-001X formulation, as expected. The gels for the 0.1% NB-00X and 0.3% NB-00X did not hinder delivery. The amount of CPC found in the receptor compartment at 24 hours was below the level of detection (5 μg/ml) for all the formulations.

At the twelve hour time point, the gel formulation delivered two-fold higher levels of CPC into the epidermis, indicating a fast rate of delivery. The dermal levels were similar (See FIG. 3).

In summary, the present invention provides a nanoemulsion for treating acne. Since the mechanism of the nanoemulsion is physical via membrane destabilization, it is unlikely to induce resistance to the nanoemulsion.

Greater than 50% of the P. acnes isolates were resistant to erythromycin and clindamycin. 44% of the isolates were resistant to tetracycline. However, single or multi-drug-resistant isolates were equally susceptible to either NB-00X or NB-00X gel. Neither NB-00X placebo had any microbiological activity. NB-00X was bactericidal against all the isolates, including isolates that were erythromycin-clindamycin- and/or tetracycline-resistant. In the absence of artificial sebum under anaerobic conditions, NB-00X has MIC₉₀/MBC₉₀ values of 0.5/2 μg/ml. Benzoyl peroxide and salicyclic acid had MIC₉₀/MBC₉₀ values of 50/200 μg/ml and 1000/2000 μg/ml, respectively.

NB-00X has relevant anti-microbiological and bactericidal activity against a collection of recent clinical isolates of P. acnes, including multidrug-resistant strains. Comparator drugs that have been used to treat acne, such as erythromycin, clindamycin, tetracycline, benzoyl peroxide and salicyclic acid were much less effective comparing to the nanoemulsion of the invention.

The MIC₉₀/MBC₉₀ values in the presence of 50% sebum increased to 128/1024 μg/ml. A reduction in the MBC₉₀ for NB-00X occurred when BPO or SA was integrated into the formulation, resulting in a MIC₉₀/MBC₉₀ of 128/256 μg/ml. This result suggests a synergy between NB-00X and benzoyl peroxide or salicylic acid.

### Example 5

### Effect of EDTA on the Efficacy of NB003 and Other Emulsions Against P. acnes in the Presence and Absence of Artificial Sebum

Background and Purpose of Study: As noted above, Propionibacterium acnes, a gram-positive, non-spore forming, anaerobic bacillus, is one of the primary factors involved in the pathogenesis of acne vulgaris. It is the predominant microorganism of the pilosebaceous glands of human skin, with up to 10 million viable organisms isolated from a single sebaceous unit. Although aerotolerant, P. acnes typically grows in the anaerobic environment of the infrainfundibulum, where it releases lipases and digests local accumulations of the skin, oil and sebum. Sebaceous glands produce an oily sebum that is primarily composed of waxes, triglycerides, and free fatty acids. Previous studies have shown that NB-00X nanodroplets are concentrated in the pilosebaceous unit where P. acnes migrates to enjoy a rich source of food (sebum) and a preferred anaerobic environment. Purpose of this study was to evaluate the efficacy to nanoemulsion against propionibacterium acnes in the presence of artificial sebum.

The efficacy of the nanoemulsions with varying concentrations of added ethylenediaminetetraacetic acid (EDTA) was evaluated using broth microdilution standard method prescribed by Clinical and Laboratory Standard Institute (CLSI). (National Committee for Clinical Laboratory Standards, “Methods for Antimicrobial Susceptibility Testing of Anaerobic Bacteria,” 7^{th} ed., Approved Standard M11-A7 (National Committee for Clinical Laboratory Standards, Wayne, Pa., 2007.) Minimum inhibitory concentration (MIC) and minimum bactericidal concentration (MBCs) of different emulsions was determined in the presence and absence of 25% artificial sebum.

Materials and Methods

Source of drugs and isolates: Emulsions tested in this study were NB-003, 10% W₂₀5 GBA2ED, and 50% S8GC. Each of these compositions is described in the table below (the composition of the neat, undiluted NB-003 formulation is given in the table below).

TABLE 9
Nanoemulsions Tested

| Nanoemulsion | Components | Weight % |
|---|---|---|
| 10% W₂₀5GBA2ED | EDTA, USP | 0.007 |
| (w/w %) | BTC 824 | 0.4 |
| | Sterile Distilled | 91.71 |
| | Water | |
| | Tween 20 | 0.592 |
| | Glycerol | 1.008 |
| | Soybean Oil | 6.279 |
| 50% S8GC | CPC | 0.535% |
| | Distilled Water | 60 |
| | Glycerol | 4% |
| | SDS | 4% |
| | Soybean Oil | 31.5 |
| NB-003 (neat) | Distilled Water | 23.42 |
| | CPC | 1.07 |
| | EDTA | 0.07 |
| | Tween 20 | 5.92 |
| | Ethanol | 6.73 |
| | Soybean oil | 62.79 |

Seven of the clinical isolates of P. acnes (PAC-004 to PAC010) used in this study were obtained from Basilea Pharmaceutica, AG, Basel, Switzerland. The majority of these isolates had defined resistance mechanisms to erythromycin, clindamycin, and/or tetracycline. Isolate numbers PAC-001 to PAC-003 were obtained from American Type Culture collection (ATCC) (Manassas, Va.).

Preparation of artificial sebum. Artificial sebum was prepared by adding the entire ingredients given in the Table 10 and heating at 60° C. in a water bath, with intermittent stirring until all solids melted to a clear yellow liquid. (Lu et al., “Comparison of artificial sebum with human and hamster sebum samples,” Int. J. Pharm., Epub date, Oct. 22, 2008.)

TABLE 10
Composition of artificial sebum

| | Vendor/ | | | Amt (g) for |
|---|---|---|---|---|
| Ingredient | Manufacturer | Catalogue # | w/w % | 200 g |
| Oleic acid | Aldrich | 364525 | 1.4 | 2.80 |
| Palmitoleic acid | VWR (Acros) | 200020-298 | 5.0 | 10.0 |
| Squalene | VWR (MP | 102948 | 15 | 30.0 |
| | Biomedical) | | | |
| Olive oil | Spectrum | OL130 | 10 | 50.0 |
| Cottonseed oil | Spectrum | CO145 | 25 | 50.0 |
| Cholesterol | JT Baker | 676-05 | 1.2 | 2.40 |
| Cholesterol oleate | Aldrich | 372935 | 2.4 | 4.80 |
| Palmitic acid | VWR | 80108-252 | 5.0 | 10.0 |
| Spermaceti wax | VWR (Aqua | 101226-030 | 15 | 30.0 |
| | Solutions) | | | |
| Paraffin wax (mp | Aldrich | 327212 | 10 | 20.0 |
| 58-62 C.) | | | | |
| Coconut oil | Aldrich | C1758 | 10 | 20.0 |

Preparation of 96-well drug plates with different concentration of EDTA. Stock solutions of drugs were prepared at 4× of first test concentration in sterile deionized water (DI water). Intermediate dilutions were prepared by 1:1 serial dilutions from stock. Final concentrations were made by 1:1 dilutions of intermediate concentrations in 2× Wilkin Chalgren media to give 2× of the final test concentrations. 50 μl of final dilutions were placed in 96 well plates. Different concentrations of EDTA were added to 96 well plates. To achieve 5 mM-20 mM of EDTA/well, 5 μl to 20 μl of 100 mM EDTA stock solution was added to each well. For 1 mM to 5 mM concentration of EDTA/well, stock solution of 100 mM was diluted 20 mM and 5 μl-25 μl of diluted stock was added into each well. Prior to inoculation, 25% of sebum was added to appropriate plates. To obtain 25% of sebum concentration, 25 μl of artificial sebum kept at 60° C. was pipetted into each well.

Determination of MICs and MBCs. P. acnes strains grown on sheep blood agar for 24-48 hrs at 35° C. were used as the sources of inocula for susceptibility studies as per CLSI. A bacterial suspension with a turbidity equivalent to a 0.5 McFarland standard was diluted to 1:10 to 1:50 in sterile saline. 5 μl to 50 μL of the adjusted inocula was added into each well to give ˜10⁶ cfu/ml after inoculation. Verification of the colony-forming units in the inoculum was performed by diluting the adjusted inoculum preparation to 10⁻⁴ and plating 100 μl on blood agar plate.

Microtiter and blood agar plates were incubated at 35-37° C. for 48 h in a 7.0 L AnaeroPack Jar (Mitsubishi gas chemical; No. 50-70) fitted with an anaerobic gas generating system (Misubishi, No. 10-01) and a dry anaerobic indicator strip (BBL, Becton, Dickinson & Co.). MICs were read visually using a 96-well plate reader fitted with a magnifying mirror (Biodesign of New York). Colony-forming units were counted after 72 h of incubation to ensure that the initial inocula were between 2-5×10⁶ cfu/ml.

The minimal bactericidal concentrations (MBC) for P. acnes were determined by plating 10 μl from the well representing the MIC plus 4 wells above the MIC on blood agar plates.

Inoculated petri plates were incubated for 72 h at 35° C. under anaerobic conditions. The MBC was calculated as the concentration of drug that gave ≧3-log reduction from the initial inoculum concentration.

Results

As shown in Table 11, the MIC of NB-003 without any EDTA ranged from 0.25 to 0.5 μg/mL. In the presence of 25% sebum, the MIC range increased to 16-32 μg/mL. With addition of 1 mM to 20 mM of EDTA, the MIC in the presence and absence of sebum decreased to ≧tested concentration of 0.063 and 1 ug/ml, respectively.

TABLE 11
MIC of NB-003 emulsions in the presence and absence of artificial
sebum

| | 0.5% | | 0.5% | | 0.5% | | 0.5% | | | |
|---|---|---|---|---|---|---|---|---|---|---|
| | NB003 + | | NB003 + | | NB003 + | | NB003 + | | | |
| | 20 mM | | 10 mM | | 5 mM | | 1 mM | | | |
| | EDTA/well | | EDTA/well | | EDTA/well | | EDTA/well | | 0.5% NB003 | |
| | No | 25% | No | 25% | No | 25% | No | 25% | No | 25% |
| PAC# | sebum | sebum | sebum | sebum | sebum | sebum | sebum | sebum | sebum | sebum |
| PAC- | ≦0.063 | ND | ≦0.063 | ND | ≦0.063 | ≦1 | ≦0.063 | ≦1 | 0.5 | 32 |
| 001 | | | | | | | | | | |
| PAC- | ≦0.063 | ND | ≦0.063 | ND | ≦0.063 | ≦1 | ≦0.063 | ≦1 | 0.25 | 16 |
| 002 | | | | | | | | | | |
| PAC- | ≦0.063 | ND | ≦0.063 | ND | ≦0.063 | ≦1 | ≦0.063 | ≦1 | 0.25 | 32 |
| 003 | | | | | | | | | | |
| PAC- | ≦0.063 | ND | ≦0.063 | ND | ≦0.063 | ≦1 | ≦0.063 | ≦1 | 0.5 | 32 |
| 004 | | | | | | | | | | |
| PAC- | ≦0.063 | ND | ≦0.063 | ND | ≦0.063 | ≦1 | ≦0.063 | ≦1 | 0.25 | 16 |
| 005 | | | | | | | | | | |
| PAC- | ≦0.063 | ND | ≦0.063 | ND | ≦0.063 | ≦1 | ≦0.063 | ≦1 | 0.5 | 16 |
| 006 | | | | | | | | | | |
| PAC- | ≦0.063 | ND | ≦0.063 | ND | ≦0.063 | ≦1 | ≦0.063 | ≦1 | 0.5 | 16 |
| 007 | | | | | | | | | | |
| PAC- | ≦0.063 | ND | ≦0.063 | ND | ≦0.063 | ≦1 | ≦0.063 | ≦1 | 0.5 | 16 |
| 008 | | | | | | | | | | |
| PAC- | ≦0.063 | ND | ≦0.063 | ND | ≦0.063 | ≦1 | ≦0.063 | ≦1 | 0.5 | 16 |
| 009 | | | | | | | | | | |
| PAC- | ≦0.063 | ND | ≦0.063 | ND | ≦0.063 | ≦1 | ≦0.063 | ≦1 | 0.25 | 16 |
| 010 | | | | | | | | | | |
| MIC | ≦0.063 | ND | ≦0.063 | ND | ≦0.063 | ≦1 | ≦0.063 | ≦1 | 0.25-0.5 | 16-32 |
| range | | | | | | | | | | |
| MIC 50 | ≦0.063 | ND | ≦0.063 | ND | ≦0.063 | ≦1 | ≦0.063 | ≦1 | 0.5 | 16 |
| MIC 90 | ≦0.063 | ND | ≦0.063 | ND | ≦0.063 | ≦1 | ≦0.063 | ≦1 | 0.5 | 32 |

MBCs data of NB-003 with varying concentration of EDTA is presented in Table 12. A review of this table shows that addition of 25% sebum increased the MBCs to 128->256 fold. The addition of EDTA decreases the MBCs in the presence of sebum. At a concentration of 10 and 20 mM of EDTA, the MBCs for all isolates were reduced to ≦tested concentration.

TABLE 12
MBCs of NB003 emulsions in the presence and absence of artificial
sebum

| | 0.5% | | 0.5% | | | | 0.5% | | | |
|---|---|---|---|---|---|---|---|---|---|---|
| | NB003 + | | NB003 + | | 0.5% | | NB003 + | | | |
| | 20 mM | | 10 mM | | NB003 + 5 mM | | 1 mM | | | |
| | EDTA/well | | EDTA/well | | EDTA/well | | EDTA/well | | 0.5% NB003 | |
| | No | 25% | No | 25% | No | 25% | No | 25% | No | 25% |
| PAC# | sebum | sebum | sebum | sebum | sebum | sebum | sebum | sebum | sebum | sebum |
| PAC- | ≦0.063 | ≦1 | ≦0.063 | ≦1 | ≦0.063 | <1 | 2 | >16 | 2 | 256 |
| 001 | | | | | | | | | | |
| PAC- | ≦0.063 | ≦1 | ≦0.063 | ≦1 | 0.125 | 1 | 2 | 16 | 2 | 512 |
| 002 | | | | | | | | | | |
| PAC- | ≦0.063 | ≦1 | ≦0.063 | ≦1 | 0.5 | 4 | 1 | 32 | 2 | 512 |
| 003 | | | | | | | | | | |
| PAC- | ≦0.063 | ≦1 | ≦0.063 | ≦1 | 0.25 | <1 | >1 | >16 | 2 | 512 |
| 004 | | | | | | | | | | |
| PAC- | ≦0.063 | ≦1 | ≦0.063 | ≦1 | ≦0.063 | 4 | >1 | 16 | 2 | 256 |
| 005 | | | | | | | | | | |
| PAC- | ≦0.063 | ≦1 | ≦0.063 | ≦1 | 0.25 | <1 | 1 | >16 | 2 | 256 |
| 006 | | | | | | | | | | |
| PAC- | ≦0.063 | ≦1 | ≦0.063 | ≦1 | 0.125 | <1 | >1 | >16 | 1 | >512 |
| 007 | | | | | | | | | | |
| PAC- | ≦0.063 | ≦1 | ≦0.063 | ≦1 | 0.125 | <1 | >1 | >16 | 2 | 256 |
| 008 | | | | | | | | | | |
| PAC- | ≦0.063 | ≦1 | ≦0.063 | ≦1 | 0.5 | <1 | >1 | >16 | 2 | 512 |
| 009 | | | | | | | | | | |
| PAC- | ≦0.063 | ≦1 | ≦0.063 | ≦1 | 0.125 | <1 | >1 | >16 | 2 | 128 |
| 010 | | | | | | | | | | |
| MBC | ≦0.063 | ≦1 | ≦0.063 | ≦1 | ≦0.063-0.5 | <1-4 | 1->1 | 16->16 | 1-2 | 128->512 |
| Range | | | | | | | | | | |
| MBC | ≦0.063 | ≦1 | ≦0.063 | ≦1 | 0.125 | <1 | >1 | >16 | 2 | 256 |
| 50 | | | | | | | | | | |
| MBC90 | ≦0.063 | ≦1 | ≦0.063 | ≦1 | 0.5 | 4 | 2 | >16 | 2 | 512 |

Table 13 shows the MICs and MBCs of Benzalkonium Chloride and SDS emulsions with addition of 20 mM EDTA to test concentrations. The trend of reduced MICs and MBCs with addition of EDTA is continued.

TABLE 13
MICs and MBCs of Selected Nanoemulsions

| | |
|---|---|
| MIC | MBC |

| | Without | With | Without | With |
|---|---|---|---|---|
| Drug | sebum | sebum | sebum | sebum |
| 10% | 0.5 | 62.5 | 1 | 62.5 |
| W₂₀5GBA₂20ED | | | | |
| 10% | ≦0.125 | ≦2 | ≦0.125 | ≦2 |
| W₂₀5GBA₂ED + | | | | |
| 20 mM | | | | |
| EDTA/well | | | | |
| 50% S8GC | 0.5 | 8 | 2 | 16 |
| 50% S8GC + | ≦0.063 | — | ≦0.063 | ≦1 |
| 20 mM | | | | |
| EDTA/well | | | | |

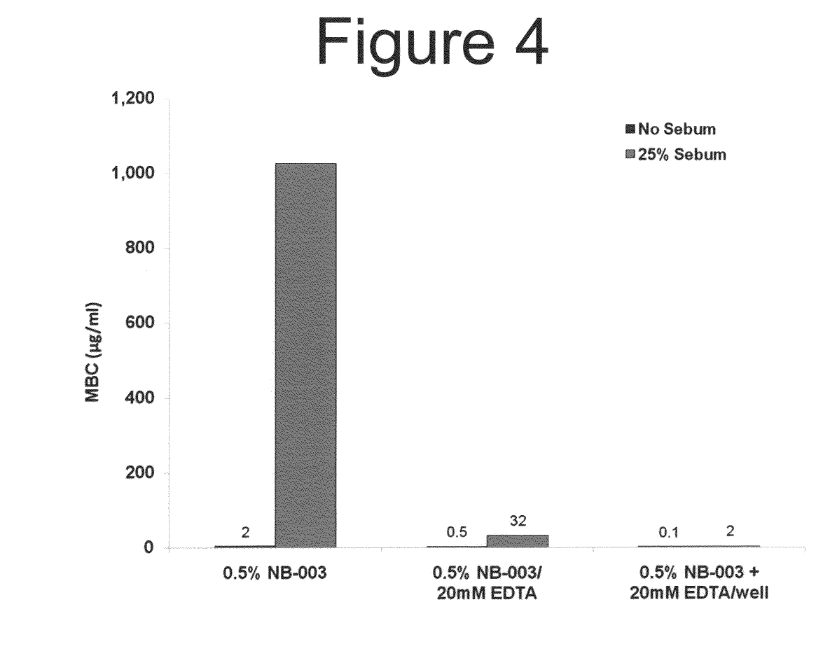

Conclusion: The MICs of a nanoemulsion according to the invention (e.g., NB-003) without any additional EDTA showed a 32 to 64 fold increase in the presence of 25% artificial sebum. MBCs of a nanoemulsion according to the invention (e.g., NB-003) showed 256 fold increases in the presence of sebum. The addition of 10-20 mM of EDTA decreased the MICs and MBCs of a nanoemulsion according to the invention (e.g., NB-003) to equal or lesser the test concentrations. See also FIG. 4, which shows the in vitro MBC of a nanoemulsion (NB-003) with and without (+/−) the presence of ethylenediaminetetraacetic acid (EDTA). The figure shows that the MBC of the nanoemulsion rises 500-fold in the presence of sebum, unless additional EDTA is added to the formulation.

### Example 6

### Viscosity

The purpose of this example was to evaluate the effect of concentration of a nanoemulsion has on the viscosity of the nanoemulsion.

FIG. 5 shows the relationship between the particle size (nm), concentration of active (%), and viscosity of a nanoemulsion. The particle size does not change upon dilution of a nanoemulsion; however viscosity significantly decreases as a function of the decrease in particle concentrations. Table 14 shows the effect dilution of a nanoemulsion has on the concentration of the active (CPC), viscosity, and particle size.

TABLE 14
NB-001 Process Optimization - Dilution

| Percentage of | Theoretical CPC | | Particle |
|---|---|---|---|
| Concentrated | Potency | Viscosity | Size |
| NB-001 | (% wt/v) | (cP) | (nm) |
| 100% | 1.0 | 259,300 | 181 |
| 80% | 0.8 | 3200 | 179 |
| 60% | 0.6 | 11.5 | 181 |
| 50% | 0.5 | 11.5 | 180 |
| 40% | 0.4 | 7.5 | 178 |
| 30% | 0.3 | 6.5 | 179 |
| 20% | 0.2 | 4.5 | 181 |
| 10% | 0.1 | 2.5 | 180 |

### Example 7

### Viscosity and Permeation

The purpose of this example was to evaluate the effect viscosity of a nanoemulsion has on the permeation of the active into the dermis and epidermis.

A permeation study was conducted using the protocol described in Example 4 with five skin sections (n=5). Four different concentrations of nanoemulsion (see Table 14) were tested: 0.25%, 0.30%, 0.50% and 0.80%. FIGS. 6 and 7 show the results for epidermis and dermis permeation, respectively. Specifically, FIG. 6 shows the results of the permeation study utilizing pig skin epidermis with 5 skin sections (n=5) following administration of a nanoemulsion (NB-003) twice daily (BID). Higher viscosity (greater than 1000 cps) nanoemulsions (e.g. 0.8% NB-003) were found to have greater permeation of the nanoemulsion into the epidermis.

Similarly, FIG. 7 shows the results of a permeation study utilizing pig skin epidermis with 5 skin sections (n=5) following administration of a nanoemulsion (NB-003) twice daily (BID). Higher viscosity (greater than 1000 cps) nanoemulsions (e.g. 0.8% NB-003) were found to deliver three times the amount of the surfactant, cetylpyridinium chloride (CPC) to the dermis as compared to a lower viscosity nanoemulsion (e.g., 0.25% NB-003).

Thus, increasing the viscosity of a nanoemulsion can increase the permeation of the nanoemulsion into the dermis and epidermis, thereby producing a composition more effective in killing bacteria or other organisms.

### Example 8

### Effect of Temperature on Nanoemulsion Effectiveness

The purpose of this example was to evaluate the effect of the temperature of the nanoemulsion on the efficacy of the nanoemulsion against P. acnes.

The effectiveness of the nanoemulsion (NB-003) in killing P. acnes over time at the three different temperatures was evaluated: 5° C., room temperature, and 37° C. The nanoemulsion was tested in the presence of 25% serum.

The results, depicted in FIG. 8, show that cooling the nanoemulsion decreases the effectiveness of the nanoemulsion in killing P. acnes. Conversely, nanoemulsions at room temperature and warmed to 37° C. showed an increased effectiveness in killing P. acnes. The nanoemulsion warmed to 37° C. showed an initial greater effectiveness in killing P. acnes as compared to the room temperature nanoemulsion, with this increase in effectiveness diminishing about 15 minutes after application. These results suggest that one tactic that may increase the effectiveness of a nanoemulsion according to the invention in treating acne is ensuring that the nanoemulsion is at room temperature or warmer prior to application.

It will be apparent to those skilled in the art that various modifications and variations can be made in the methods and compositions of the present invention without departing from the spirit or scope of the invention. Thus, it is intended that the present invention cover the modifications and variations of this invention provided they come within the scope of the appended claims and their equivalents.

What is claimed is:

1. A method of killing P. acnes, in a subject in need thereof comprising administering topically to the subject a nanoemulsion, wherein:
(a) the nanoemulsion comprises droplets having an average diameter of less than about 3 microns; and
(b) the nanoemulsion droplets comprise an oil phase with at least one oil, an aqueous phase comprising at least one surfactant, at least one organic solvent, and water.

2. The method of claim 1, wherein the nanoemulsion droplets target the pilosebaceous gland.

3. The method of claim 1, wherein the nanoemulsion has a viscosity selected from the group consisting of greater than about 12 centipoise (cP), greater than about 15 cP, greater than about 20 cP, greater than about 25 cP, greater than about 30 cP, greater than about 35 cP, greater than about 40 cP, greater than about 45 cP, greater than about 50 cP, greater than about 55 cP, greater than about 60 cP, greater than about 65 cP, greater than about 70 cP, greater than about 75 cP, greater than about 80 cP, greater than about 85 cP, greater than about 90 cP, greater than about 95 cP, greater than about 100 cP, greater than about 150 cP, greater than about 200 cP, greater than about 300 cP, greater than about 400 cP, greater than about 500 cP, greater than about 600 cP, greater than about 700 cP, greater than about 800 cP, greater than about 900 cP, greater than about 1000 cP, greater than about 1500 cP, greater than about 2000 cP, greater than about 2500 cP, greater than about 3000 cP, greater than about 3500 cP, greater than about 4000 cP, greater than about 4500 cP, greater than about 5000 cP, greater than about 5500 cP, greater than about 6000 cP, greater than about 7000 cP, greater than about 8000 cP, greater than about 9000 cP, greater than about 10,000 cP, greater than about 15,000 cP, greater than about 20,000 cP, greater than about 30,000 cP, greater than about 40,000 cP, greater than about 50,000 cP, greater than about 60,000 cP, greater than about 70,000 cP, greater than about 80,000 cP, greater than about 90,000 cP, greater than about 100,000 cP, greater than about 150,000 cP, greater than about 200,000 cP, greater than about 250,000 cP, or up to about 259,300 cP.

4. The method of claim 1, wherein the nanoemulsion is at room temperature at the time of administration.

5. The method of claim 1, wherein prior to application the nanoemulsion is warmed to a temperature selected from the group consisting of about 30° C. or warmer, about 31° C. or warmer, about 32° C. or warmer, about 33° C. or warmer, about 34° C. or warmer, about 35° C. or warmer, about 36° C. or warmer, and about 37° C.

6. The method of claim 1, wherein:
(a) the nanoemulsion droplets have an average diameter selected from the group consisting of less than about 950 nm, less than about 900 nm, less than about 850 nm, less than about 800 nm, less than about 750 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 200 nm, less than about 150 nm, less than about 100 nm, greater than about 50 nm, greater than about 70 nm, greater than about 125 nm, and any combination thereof;
(b) the nanoemulsion droplets have an average diameter greater than about 125 nm and less than about 450 nm; or
(c) any combination thereof.

7. The method of claim 1, wherein the topical application is to any superficial skin structure.

8. The method of claim 1, wherein the nanoemulsion further comprises a chelating agent.

9. The method of claim 8, wherein the chelating agent:
(a) is present in an amount of about 0.0005% to about 1.0%:
(b) is selected from the group consisting of ethylenediamine, ethylenediaminetetraacetic acid, and dimercaprol; or
(c) any combination thereof.

10. The method of claim 1, wherein the nanoemulsion comprises:
(a) an aqueous phase;
(b) about 1% oil to about 80% oil;
(c) about 0.1% organic solvent to about 50% organic solvent;
(d) at least one surfactant present in an amount of about 0.001% surfactant to about 10% surfactant;
(e) about 0.0005% to about 1.0% of a chelating agent; or
(f) any combination thereof.

11. The method of claim 1, wherein the nanoemulsion comprises:
(a) an aqueous phase;
(b) about 5% oil to about 80% oil;
(c) about 0.1% organic solvent to about 10% organic solvent;
(d) at least one non-ionic surfactant present in an amount of about 0.1% to about 10%;
(e) at least one cationic agent present in an amount of about 0.01% to about 2%;
(f) about 0.0005% to about 1.0% of a chelating agent; or
(g) any combination thereof.

12. The method of claim 1, wherein:
(a) the nanoemulsion is stable at about 40° C. and about 75% relative humidity for a time period selected from the group consisting of up to about 1 month, up to about 3 months, up to about 6 months, up to about 12 months, up to about 18 months, up to about 2 years, up to about 2.5 years, and up to about 3 years;
(b) the nanoemulsion is stable at about 25° C. and about 60% relative humidity for a time period selected from the group consisting of up to about 1 month, up to about 3 months, up to about 6 months, up to about 12 months, up to about 18 months, up to about 2 years, up to about 2.5 years, up to about 3 years, up to about 3.5 years, up to about 4 years, up to about 4.5 years, and up to about 5 years;
(c) the nanoemulsion is stable at about 4° C. for a time period selected from the group consisting of up to about 1 month, up to about 3 months, up to about 6 months, up to about 12 months, up to about 18 months, up to about 2 years, up to about 2.5 years, up to about 3 years, up to about 3.5 years, up to about 4 years, up to about 4.5 years, up to about 5 years, up to about 5.5 years, up to about 6 years, up to about 6.5 years, and up to about 7 years; or
(d) any combination thereof.

13. The method of claim 1, wherein the organic solvent:
(a) is selected from the group consisting of C₁-C₁₂ alcohol, diol, triol, dialkyl phosphate, tri-alkyl phosphate, semi-synthetic derivatives thereof, and combinations thereof;
(b) is an alcohol which is selected from the group consisting of a nonpolar solvent, a polar solvent, a protic solvent, and an aprotic solvent;
(c) is selected from the group consisting of ethanol, methanol, isopropyl alcohol, glycerol, medium chain triglycerides, diethyl ether, ethyl acetate, acetone, dimethyl sulfoxide (DMSO), acetic acid, n-butanol, butylene glycol, perfumers alcohols, isopropanol, n-propanol, formic acid, propylene glycols, glycerol, sorbitol, industrial methylated spirit, triacetin, hexane, benzene, toluene, diethyl ether, chloroform, 1,4-dixoane, tetrahydrofuran, dichloromethane, acetone, acetonitrile, dimethylformamide, dimethyl sulfoxide, formic acid, semi-synthetic derivatives thereof, and any combination thereof; or
(d) any combination thereof.

14. The method of claim 13, wherein the tri-alkyl phosphate is tri-n-butyl phosphate.

15. The method of claim 1, wherein the oil:
(a) is any cosmetically or pharmaceutically acceptable oil:
(b) is non-volatile;
(c) is selected from the group consisting of animal oil, vegetable oil, natural oil, synthetic oil, hydrocarbon oils, silicone oils, and semi-synthetic derivatives thereof;
(d) is selected from the group consisting of mineral oil, squalene oil, flavor oils, silicon oil, essential oils, water insoluble vitamins, Isopropyl stearate, Butyl stearate, Octyl palmitate, Cetyl palmitate, Tridecyl behenate, Diisopropyl adipate, Dioctyl sebacate, Menthyl anthranhilate, Cetyl octanoate, Octyl salicylate, Isopropyl myristate, neopentyl glycol dicarpate cetols, Ceraphyls®, Decyl oleate, diisopropyl adipate, C₁₂₋₁₅ alkyl lactates, Cetyl lactate, Lauryl lactate, Isostearyl neopentanoate, Myristyl lactate, Isocetyl stearoyl stearate, Octyldodecyl stearoyl stearate, Hydrocarbon oils, Isoparaffin, Fluid paraffins, Isododecane, Petrolatum, Argan oil, Canola oil, Chile oil, Coconut oil, corn oil, Cottonseed oil, Flaxseed oil, Grape seed oil, Mustard oil, Olive oil, Palm oil, Palm kernel oil, Peanut oil, Pine seed oil, Poppy seed oil, Pumpkin seed oil, Rice bran oil, Safflower oil, Tea oil, Truffle oil, Vegetable oil, Apricot (kernel) oil, Jojoba oil (simmondsia chinensis seed oil), Grapeseed oil, Macadamia oil, Wheat germ oil, Almond oil, Rapeseed oil, Gourd oil, Soybean oil, Sesame oil, Hazelnut oil, Maize oil, Sunflower oil, Hemp oil, Bois oil, Kuki nut oil, Avocado oil, Walnut oil, Fish oil, berry oil, allspice oil, juniper oil, seed oil, almond seed oil, anise seed oil, celery seed oil, cumin seed oil, nutmeg seed oil, leaf oil, basil leaf oil, bay leaf oil, cinnamon leaf oil, common sage leaf oil, eucalyptus leaf oil, lemon grass leaf oil, melaleuca leaf oil, oregano leaf oil, patchouli leaf oil, peppermint leaf oil, pine needle oil, rosemary leaf oil, spearmint leaf oil, tea tree leaf oil, thyme leaf oil, wintergreen leaf oil, flower oil, chamomile oil, clary sage oil, clove oil, geranium flower oil, hyssop flower oil, jasmine flower oil, lavender flower oil, manuka flower oil, Marhoram flower oil, orange flower oil, rose flower oil, ylang-ylang flower oil, Bark oil, cassia Bark oil, cinnamon bark oil, sassafras Bark oil, Wood oil, camphor wood oil, cedar wood oil, rosewood oil, sandalwood oil), rhizome (ginger) wood oil, resin oil, frankincense oil, myrrh oil, peel oil, bergamot peel oil, grapefruit peel oil, lemon peel oil, lime peel oil, orange peel oil, tangerine peel oil, root oil, valerian oil, Oleic acid, Linoleic acid, Oleyl alcohol, Isostearyl alcohol, semi-synthetic derivatives thereof, and combinations thereof; or
(e) any combination thereof.

16. The method of claim 1, further comprising a silicone component.

17. The method of claim 16, wherein the silicone component comprises at least one volatile silicone oil, wherein:
(a) the volatile silicone oil can be the sole oil in the silicone component or it can be combined with other silicone and non-silicone oils, and wherein the other oils can be volatile or non-volatile;
(b) the volatile oil used in the silicone component is different than the oil in the oil phase;
(c) the silicone component is selected from the group consisting of methylphenylpolysiloxane, simethicone, dimethicone, phenyltrimethicone (or an organomodified version thereof), alkylated derivatives of polymeric silicones, cetyl dimethicone, lauryl trimethicone, hydroxylated derivatives of polymeric silicones, such as dimethiconol, volatile silicone oils, cyclic and linear silicones, cyclomethicone, derivatives of cyclomethicone, hexamethylcyclotrisiloxane, octamethylcyclotetrasiloxane, decamethylcyclopentasiloxane, volatile linear dimethylpolysiloxanes, isohexadecane, isoeicosane, isotetracosane, polyisobutene, isooctane, isododecane, semi-synthetic derivatives thereof, and combinations thereof; or
(d) any combination thereof.

18. The method of claim 1, wherein the nanoemulsion comprises a volatile oil, wherein:
(a) the volatile oil can be the organic solvent, or the volatile oil can be present in addition to an organic solvent;
(b) the volatile oil is a terpene, monoterpene, sesquiterpene, carminative, azulene, semi-synthetic derivatives thereof, or combinations thereof;
(c) the volatile oil is selected from the group consisting of a terpene, monoterpene, sesquiterpene, carminative, azulene, menthol, camphor, thujone, thymol, nerol, linalool, limonene, geraniol, perillyl alcohol, nerolidol, farnesol, ylangene, bisabolol, farnesene, ascaridole, chenopodium oil, citronellal, citral, citronellol, chamazulene, yarrow, guaiazulene, chamomile, semi-synthetic derivatives thereof, and combinations thereof; or
(d) any combination thereof.

19. The method of claim 1, wherein the surfactant is:
(a) a pharmaceutically acceptable ionic surfactant, a pharmaceutically acceptable nonionic surfactant, a pharmaceutically acceptable cationic surfactant, a pharmaceutically acceptable ionic surfactant, a pharmaceutically acceptable anionic surfactant, or a pharmaceutically acceptable zwitterionic surfactant;
(b) a pharmaceutically acceptable ionic polymeric surfactant, a pharmaceutically acceptable nonionic polymeric surfactant, a pharmaceutically acceptable cationic polymeric surfactant, a pharmaceutically acceptable anionic polymeric surfactant, or a pharmaceutically acceptable zwitterionic polymeric surfactant;
(c) a polymeric surfactant which is selected from the group consisting of a graft copolymer of a poly(methyl methacrylate) backbone with at least one polyethylene oxide (PEO) side chain, polyhydroxystearic acid, an alkoxylated alkyl phenol formaldehyde condensate, a polyalkylene glycol modified polyester with fatty acid hydrophobes, a polyester, semi-synthetic derivatives thereof, and combinations thereof;
(d) selected from the group consisting of ethoxylated nonylphenol comprising 9 to 10 units of ethyleneglycol, ethoxylated undecanol comprising 8 units of ethyleneglycol, polyoxyethylene (20) sorbitan monolaurate, polyoxyethylene (20) sorbitan monopalmitate, polyoxyethylene (20) sorbitan monostearate, polyoxyethylene (20) sorbitan monooleate, sorbitan monolaurate, sorbitan monopalmitate, sorbitan monostearate, sorbitan monooleate, ethoxylated hydrogenated ricin oils, sodium laurylsulfate, a diblock copolymer of ethyleneoxyde and propyleneoxyde, Ethylene Oxide-Propylene Oxide Block Copolymers, and tetra-functional block copolymers based on ethylene oxide and propylene oxide, Glyceryl monoesters, Glyceryl caprate, Glyceryl caprylate, Glyceryl cocate, Glyceryl erucate, Glyceryl hydroxysterate, Glyceryl isostearate, Glyceryl lanolate, Glyceryl laurate, Glyceryl linolate, Glyceryl myristate, Glyceryl oleate, Glyceryl PABA, Glyceryl palmitate, Glyceryl ricinoleate, Glyceryl stearate, Glyceryl thighlycolate, Glyceryl dilaurate, Glyceryl dioleate, Glyceryl dimyristate, Glyceryl disterate, Glyceryl sesuioleate, Glyceryl stearate lactate, Polyoxyethylene cetyl/stearyl ether, Polyoxyethylene cholesterol ether, Polyoxyethylene laurate or dilaurate, Polyoxyethylene stearate or distearate, polyoxyethylene fatty ethers, Polyoxyethylene lauryl ether, Polyoxyethylene stearyl ether, polyoxyethylene myristyl ether, a steroid, Cholesterol, Betasitosterol, Bisabolol, fatty acid esters of alcohols, isopropyl myristate, Aliphati-isopropyl n-butyrate, Isopropyl n-hexanoate, Isopropyl n-decanoate, Isoproppyl palmitate, Octyldodecyl myristate, alkoxylated alcohols, alkoxylated acids, alkoxylated amides, alkoxylated sugar derivatives, alkoxylated derivatives of natural oils and waxes, polyoxyethylene polyoxypropylene block copolymers, nonoxynol-14, PEG-8 laurate, PEG-6 Cocoamide, PEG-20 methylglucose sesquistearate, PEG40 lanolin, PEG-40 castor oil, PEG-40 hydrogenated castor oil, polyoxyethylene fatty ethers, glyceryl diesters, polyoxyethylene stearyl ether, polyoxyethylene myristyl ether, and polyoxyethylene lauryl ether, glyceryl dilaurate, glyceryl dimystate, glyceryl distearate, semi-synthetic derivatives thereof, and mixtures thereof;
(e) a non-ionic lipid selected from the group consisting of glyceryl laurate, glyceryl myristate, glyceryl dilaurate, glyceryl dimyristate, semi-synthetic derivatives thereof, and mixtures thereof;
(f) a polyoxyethylene fatty ether having a polyoxyethylene head group ranging from about 2 to about 100 groups;
(g) an alkoxylated alcohol having the structure shown in formula I below:
R₅—(OCH₂CH₂)_{y}—OH Formula I
wherein R₅ is a branched or unbranched alkyl group having from about 6 to about 22 carbon atoms and y is between about 4 and about 100, and preferably, between about 10 and about 100;
(h) an alkoxylated alcohol which is an ethoxylated derivative of lanolin alcohol;
(i) is nonionic and is selected from the group consisting of
nonoxynol-9,
an ethoxylated surfactant,
an alcohol ethoxylated,
an alkyl phenol ethoxylated,
a fatty acid ethoxylated,
a monoalkaolamide ethoxylated,
a sorbitan ester ethoxylated,
a fatty amino ethoxylated,
an ethylene oxide-propylene oxide copolymer,
Bis(polyethylene glycol bis[imidazoyl carbonyl]),
Brij® 35,
Brij® 56,
Brij® 72,
Brij® 76,
Brij® 92V,
Brij® 97,
Brij® 58P,
Cremophor® EL,
Decaethylene glycol monododecyl ether,
N-Decanoyl-N-methylglucamine,
n-Decyl alpha-D-glucopyranoside,
Decyl beta-D-maltopyranoside,
n-Dodecanoyl-N-methylglucamide,
n-Dodecyl alpha-D-maltoside,
n-Dodecyl beta-D-maltoside,
Heptaethylene glycol monodecyl ether,
Heptaethylene glycol monotetradecyl ether,
Heptaethylene glycol monododecyl ether,
n-Hexadecyl beta-D-maltoside,
Hexaethylene glycol monododecyl ether,
Hexaethylene glycol monohexadecyl ether,
Hexaethylene glycol monooctadecyl ether,
Hexaethylene glycol monotetradecyl ether,
Igepal CA-630,
Methyl-6-O-(N-heptylcarbamoyl)-alpha-D-glucopyranoside,
Nonaethylene glycol monododecyl ether,
N-Nonanoyl-N-methylglucamine,
Octaethylene glycol monodecyl ether,
Octaethylene glycol monododecyl ether,
Octaethylene glycol monohexadecyl ether,
Octaethylene glycol monooctadecyl ether,
Octaethylene glycol monotetradecyl ether,
Octyl-beta-D-glucopyranoside,
Pentaethylene glycol monodecyl ether,
Pentaethylene glycol monododecyl ether,
Pentaethylene glycol monohexadecyl ether,
Pentaethylene glycol monohexyl ether,
Pentaethylene glycol monooctadecyl ether,
Pentaethylene glycol monooctyl ether,
Polyethylene glycol diglycidyl ether,
Polyethylene glycol ether W-1,
Polyoxyethylene 10 tridecyl ether,
Polyoxyethylene 100 stearate,
Polyoxyethylene 20 isohexadecyl ether,
Polyoxyethylene 20 oleyl ether,
Polyoxyethylene 40 stearate,
Polyoxyethylene 50 stearate,
Polyoxyethylene 8 stearate,
Polyoxyethylene bis(imidazolyl carbonyl),
Polyoxyethylene 25 propylene glycol stearate,
Saponin from Quillaja bark,
Span® 20,
Span® 40,
Span® 60,
Span® 65,
Span® 80,
Span® 85,
Tergitol,
Tergitol, Type 15-S-12,
Tergitol, Type 15-S-30,
Tergitol, Type 15-S-5,
Tergitol, Type 15-S-7,
Tergitol, Type 15-S-9,
Tergitol, Type NP-10,
Tergitol, Type NP-4,
Tergitol, Type NP-40,
Tergitol, Type NP-7,
Tergitol, Type NP-9,
Tergitol, Type TMN-10,
Tergitol, Type TMN-6,
Tetradecyl-beta-D-maltoside,
Tetraethylene glycol monodecyl ether,
Tetraethylene glycol monododecyl ether,
Tetraethylene glycol monotetradecyl ether,
Triethylene glycol monodecyl ether,
Triethylene glycol monododecyl ether,
Triethylene glycol monohexadecyl ether,
Triethylene glycol monooctyl ether,
Triethylene glycol monotetradecyl ether,
Triton CF-21,
Triton CF-32,
Triton DF-12,
Triton DF-16,
Triton GR-5M,
Triton QS-15,
Triton QS-44,
Triton X-100,
Triton X-102,
Triton X-15,
Triton X-151,
Triton X-200,
Triton X-207,
Triton X-114,
Triton X-165,
Triton X-305,
Triton X-405,
Triton X-45,
Triton X-705-70,
TWEEN® 20,
TWEEN® 21,
TWEEN® 40,
TWEEN® 60,
TWEEN® 61,
TWEEN® 65,
TWEEN® 80,
TWEEN® 81,
TWEEN® 85,
Tyloxapol
n-Undecyl beta-D-glucopyranoside,
Poloxamer 101,
Poloxamer 105,
Poloxamer 108,
Poloxamer 122,
Poloxamer 123,
Poloxamer 124,
Poloxamer 181,
Poloxamer 182,
Poloxamer 183,
Poloxamer 184,
Poloxamer 185,
Poloxamer 188,
Poloxamer 212,
Poloxamer 215,
Poloxamer 217,
Poloxamer 231,
Poloxamer 234,
Poloxamer 235,
Poloxamer 237,
Poloxamer 238,
Poloxamer 282,
Poloxamer 284,
Poloxamer 288,
Poloxamer 331,
Poloxamer 333,
Poloxamer 334,
Poloxamer 335,
Poloxamer 338,
Poloxamer 401,
Poloxamer 402,
Poloxamer 403,
Poloxamer 407,
Poloxamer 105 Benzoate,
Poloxamer 182 Dibenzoate,
semi-synthetic derivatives thereof, and combinations thereof;
(j) the surfactant is cationic and is selected from the group consisting of
a quarternary ammonium compound,
an alkyl trimethyl ammonium chloride compound,
a dialkyl dimethyl ammonium chloride compound,
Benzalkonium chloride,
Benzyldimethylhexadecylammonium chloride,
Benzyldimethyltetradecylammonium chloride,
Benzyldodecyldimethylammonium bromide,
Benzyltrimethylammonium tetrachloroiodate,
cetylpyridinium chloride,
Dimethyldioctadecylammonium bromide,
Dodecylethyldimethylammonium bromide,
Dodecyltrimethylammonium bromide,
Ethylhexadecyldimethylammonium bromide,
Girard's reagent T,
Hexadecyltrimethylammonium bromide,
N,N′,N′-Polyoxyethylene(10)-N-tallow-1,3-diaminopropane,
Thonzonium bromide,
Trimethyl(tetradecyl)ammonium bromide,
1,3,5-Triazine-1,3,5(2H,4H,6H)-triethanol,
1-Decanaminium, N-decyl-N,N-dimethyl-, chloride,
Didecyl dimethyl ammonium chloride,
2-(2-(p-(Diisobutyl)cresosxy)ethoxy)ethyl dimethyl benzyl ammonium chloride,
2-(2-(p-(Diisobutyl)phenoxy)ethoxy)ethyl dimethyl benzyl ammonium chloride,
Alkyl 1 or 3 benzyl-1-(2-hydroxethyl)-2-imidazolinium chloride,
Alkyl bis(2-hydroxyethyl)benzyl ammonium chloride,
Alkyl demethyl benzyl ammonium chloride,
Alkyl dimethyl 3,4-dichlorobenzyl ammonium chloride (100% C12),
Alkyl dimethyl 3,4-dichlorobenzyl ammonium chloride (50% C14, 40% C12, 10% C16),
Alkyl dimethyl 3,4-dichlorobenzyl ammonium chloride (55% C14, 23% C12, 20% C16),
Alkyl dimethyl benzyl ammonium chloride,
Alkyl dimethyl benzyl ammonium chloride (100% C14),
Alkyl dimethyl benzyl ammonium chloride (100% C16),
Alkyl dimethyl benzyl ammonium chloride (41% C14, 28% C12),
Alkyl dimethyl benzyl ammonium chloride (47% C12, 18% C14),
Alkyl dimethyl benzyl ammonium chloride (55% C16, 20% C14),
Alkyl dimethyl benzyl ammonium chloride (58% C14, 28% C16),
Alkyl dimethyl benzyl ammonium chloride (60% C14, 25% C12),
Alkyl dimethyl benzyl ammonium chloride (61% C11, 23% C14),
Alkyl dimethyl benzyl ammonium chloride (61% C12, 23% C14),
Alkyl dimethyl benzyl ammonium chloride (65% C12, 25% C14),
Alkyl dimethyl benzyl ammonium chloride (67% C12, 24% C14),
Alkyl dimethyl benzyl ammonium chloride (67% C12, 25% C14),
Alkyl dimethyl benzyl ammonium chloride (90% C14, 5% C12),
Alkyl dimethyl benzyl ammonium chloride (93% C14, 4% C12),
Alkyl dimethyl benzyl ammonium chloride (95% C16, 5% C18),
Alkyl didecyl dimethyl ammonium chloride,
Alkyl dimethyl benzyl ammonium chloride (C12-16),
Alkyl dimethyl benzyl ammonium chloride (C12-18),
dialkyl dimethyl benzyl ammonium chloride,
Alkyl dimethyl dimethybenzyl ammonium chloride,
Alkyl dimethyl ethyl ammonium bromide (90% C14, 5% C16, 5% C12),
Alkyl dimethyl ethyl ammonium bromide (mixed alkyl and alkenyl groups as in the fatty acids of soybean oil),
Alkyl dimethyl ethylbenzyl ammonium chloride,
Alkyl dimethyl ethylbenzyl ammonium chloride (60% C14),
Alkyl dimethyl isopropylbenzyl ammonium chloride (50% C12, 30% C14, 17% C16, 3% C18),
Alkyl trimethyl ammonium chloride (58% C18, 40% C16, 1% C14, 1% C12),
Alkyl trimethyl ammonium chloride (90% C18, 10% C16),
Alkyldimethyl(ethylbenzyl) ammonium chloride (C12-18),
Di-(C8-10)-alkyl dimethyl ammonium chlorides,
Dialkyl dimethyl ammonium chloride,
Dialkyl methyl benzyl ammonium chloride,
Didecyl dimethyl ammonium chloride,
Diisodecyl dimethyl ammonium chloride,
Dioctyl dimethyl ammonium chloride,
Dodecyl bis(2-hydroxyethyl) octyl hydrogen ammonium chloride,
Dodecyl dimethyl benzyl ammonium chloride,
Dodecylcarbamoyl methyl dimethyl benzyl ammonium chloride,
Heptadecyl hydroxyethylimidazolinium chloride,
Hexahydro-1,3,5-tris(2-hydroxyethyl)-s-triazine,
Myristalkonium chloride (and) Quat RNIUM 14,
N,N-Dimethyl-2-hydroxypropylammonium chloride polymer,
n-Tetradecyl dimethyl benzyl ammonium chloride monohydrate,
Octyl decyl dimethyl ammonium chloride,
Octyl dodecyl dimethyl ammonium chloride,
Octyphenoxyethoxyethyl dimethyl benzyl ammonium chloride,
Oxydiethylenebis(alkyl dimethyl ammonium chloride),
Trimethoxysily propyl dimethyl octadecyl ammonium chloride,
Trimethoxysilyl quats,
Trimethyl dodecylbenzyl ammonium chloride,
semi-synthetic derivatives thereof, and combinations thereof;
(k) the surfactant is anionic and is selected from the group consisting of
a carboxylate,
a sulphate,
a sulphonate,
a phosphate,
Chenodeoxycholic acid,
Chenodeoxycholic acid sodium salt,
Cholic acid, ox or sheep bile,
Dehydrocholic acid,
Deoxycholic acid,
Deoxycholic acid methyl ester,
Digitonin,
Digitoxigenin,
N,N-Dimethyldodecylamine N-oxide,
Docusate sodium salt,
Glycochenodeoxycholic acid sodium salt,
Glycocholic acid hydrate, synthetic,
Glycocholic acid sodium salt hydrate, synthetic,
Glycodeoxycholic acid monohydrate,
Glycodeoxycholic acid sodium salt,
Glycolithocholic acid 3-sulfate disodium salt,
Glycolithocholic acid ethyl ester,
N-Lauroylsarcosine sodium salt,
N-Lauroylsarcosine solution,
Lithium dodecyl sulfate,
Lugol solution,
Niaproof 4, Type 4,
1-Octanesulfonic acid sodium salt,
Sodium 1-butanesulfonate,
Sodium 1-decanesulfonate,
Sodium 1-dodecanesulfonate,
Sodium 1-heptanesulfonate anhydrous,
Sodium 1-nonanesulfonate,
Sodium 1-propanesulfonate monohydrate,
Sodium 2-bromoethanesulfonate,
Sodium cholate hydrate,
Sodium choleate,
Sodium deoxycholate,
Sodium deoxycholate monohydrate,
Sodium dodecyl sulfate,
Sodium hexanesulfonate anhydrous,
Sodium octyl sulfate,
Sodium pentanesulfonate anhydrous,
Sodium taurocholate,
Taurochenodeoxycholic acid sodium salt,
Taurodeoxycholic acid sodium salt monohydrate,
Taurohyodeoxycholic acid sodium salt hydrate,
Taurolithocholic acid 3-sulfate disodium salt,
Tauroursodeoxycholic acid sodium salt,
Trizma® dodecyl sulfate,
Ursodeoxycholic acid,
semi-synthetic derivatives thereof, and combinations thereof;
(l) the surfactant is zwitterionic and is selected from the group consisting of
an N-alkyl betaine,
lauryl amindo propyl dimethyl betaine,
an alkyl dimethyl glycinate,
an N-alkyl amino propionate,
CHAPS, minimum 98%,
CHAPS, minimum 98%,
CHAPS, for electrophoresis, minimum 98%,
CHAPSO, minimum 98%,
CHAPSO,
CHAPSO, for electrophoresis,
3-(Decyldimethylammonio)propanesulfonate inner salt,
3-(Dodecyldimethylammonio)propanesulfonate inner salt,
3-(N,N-Dimethylmyristylammonio)propanesulfonate inner salt,
3-(N,N-Dimethyloctadecylammonio)propanesulfonate,
3-(N,N-Dimethyloctylammonio)propanesulfonate inner salt,
3-(N,N-Dimethylpalmitylammonio)propanesulfonate,
semi-synthetic derivatives thereof, and combinations thereof; or
(m) any combination thereof.

20. The method of claim 19, wherein:
(a) the alkoxylated alcohol is the species wherein R₅ is a lauryl group and y has an average value of 23;
(b) the ethoxylated derivative of lanolin alcohol is laneth-10, which is the polyethylene glycol ether of lanolin alcohol with an average ethoxylation value of 10.

21. The method of claim 1, wherein the nanoemulsion comprises at least one cationic surfactant.

22. The method of claim 1, wherein the nanoemulsion comprises a cationic surfactant which is cetylpyridinium chloride.

23. The method of claim 1, wherein the nanoemulsion comprises a cationic surfactant, and wherein:
(a) the concentration of the cationic surfactant is less than about 5.0% and greater than about 0.001%;
(b) the concentration of the cationic surfactant is selected from the group consisting of less than about 5%, less than about 4.5%, less than about 4.0%, less than about 3.5%, less than about 3.0%, less than about 2.5%, less than about 2.0%, less than about 1.5%, less than about 1.0%, less than about 0.90%, less than about 0.80%, less than about 0.70%, less than about 0.60%, less than about 0.50%, less than about 0.40%, less than about 0.30%, less than about 0.20%, less than about 0.10%, greater than about 0.002%, greater than about 0.003%, greater than about 0.004%, greater than about 0.005%, greater than about 0.006%, greater than about 0.007%, greater than about 0.008%, greater than about 0.009%, greater than about 0.010%, and greater than about 0.001%; or
(c) any combination thereof.

24. The method of claim 1, wherein the nanoemulsion comprises at least one cationic surfactant and at least one non-cationic surfactant.

25. The method of claim 24, wherein:
(a) the non-cationic surfactant is a nonionic surfactant;
(b) the non-cationic surfactant is a nonionic surfactant which is a polysorbate;
(c) the non-cationic surfactant is a nonionic surfactant which is polysorbate 20 or polysorbate 80 or polysorbate 60;
(d) the non-cationic surfactant is a nonionic surfactant and the non-ionic surfactant is present in a concentration of about 0.05% to about 7.0%;
(e) the non-cationic surfactant is a nonionic surfactant and the non-ionic surfactant is present in a concentration of about 0.5% to about 4%; or
(f) any combination thereof.

26. The method of claim 1, wherein the nanoemulsion comprises a cationic surfactant present in a concentration of about 0.5% to about 2%, in combination with a nonionic surfactant.

27. The method of claim 1, wherein the nanoemulsion further comprises:
(a) at least one preservative;
(b) at least one a pH adjuster;
(c) at least pharmaceutically acceptable buffer; or
(d) any combination thereof.

28. The method of claim 27, wherein:
(a) the preservative is selected from the group consisting of cetylpyridinium chloride, benzalkonium chloride, benzyl alcohol, chlorhexidine, imidazolidinyl urea, phenol, potassium sorbate, benzoic acid, bronopol, chlorocresol, paraben esters, phenoxyethanol, sorbic Acid, alpha-tocophernol, ascorbic acid, ascorbyl palmitate, butylated hydroxyanisole, butylated hydroxytoluene, sodium ascorbate, sodium metabisulphite, citric acid, edetic acid, semi-synthetic derivatives thereof, and combinations thereof;
(b) the pH adjuster is selected from the group consisting of diethyanolamine, lactic acid, monoethanolamine, triethylanolamine, sodium hydroxide, sodium phosphate, semi-synthetic derivatives thereof, and combinations thereof;
(c) the buffer is selected from the group consisting of
2-Amino-2-methyl-1,3-propanediol, ≧99.5% (NT),
2-Amino-2-methyl-1-propanol, ≧99.0% (GC),
L-(+)-Tartaric acid, ≧99.5% (T),
ACES, ≧99.5% (T),
ADA, ≧99.0% (T),
Acetic acid, ≧99.5% (GC/T),
Acetic acid, for luminescence, ≧99.5% (GC/T),
Ammonium acetate solution, for molecular biology, ˜5 M in H₂O,
Ammonium acetate, for luminescence, ≧99.0% (calc. on dry substance, T),
Ammonium bicarbonate, ≧99.5% (T),
Ammonium citrate dibasic, ≧99.0% (T),
Ammonium formate solution, 10 M in H₂O,
Ammonium formate, ≧99.0% (calc. based on dry substance, NT),
Ammonium oxalate monohydrate, ≧99.5% (RT),
Ammonium phosphate dibasic solution, 2.5 M in H₂O,
Ammonium phosphate dibasic, ≧99.0% (T),
Ammonium phosphate monobasic solution, 2.5 M in H₂O,
Ammonium phosphate monobasic, ≧99.5% (T),
Ammonium sodium phosphate dibasic tetrahydrate, ≧99.5% (NT),
Ammonium sulfate solution, for molecular biology, 3.2 M in H₂O,
Ammonium tartrate dibasic solution, 2 M in H₂O (colorless solution at 20° C.),
Ammonium tartrate dibasic, ≧99.5% (T),
BES buffered saline, for molecular biology, 2× concentrate,
BES, ≧99.5% (T),
BES, for molecular biology, ≧99.5% (T),
BICINE buffer Solution, for molecular biology, 1 M in H₂O,
BICINE, ≧99.5% (T),
BIS-TRIS, ≧99.0% (NT),
Bicarbonate buffer solution, >0.1 M Na₂CO₃, >0.2 M NaHCO₃,
Boric acid, ≧99.5% (T),
Boric acid, for molecular biology, ≧99.5% (T),
CAPS, ≧99.0% (TLC),
CHES, ≧99.5% (T),
Calcium acetate hydrate, ≧99.0% (calc. on dried material, KT),
Calcium carbonate, precipitated, ≧99.0% (KT),
Calcium citrate tribasic tetrahydrate, ≧98.0% (calc. on dry substance, KT),
Citrate Concentrated Solution, for molecular biology, 1 M in H₂O,
Citric acid, anhydrous, ≧99.5% (T),
Citric acid, for luminescence, anhydrous, ≧99.5% (T),
Diethanolamine, ≧99.5% (GC),
EPPS, ≧99.0% (T),
Ethylenediaminetetraacetic acid disodium salt dihydrate, for molecular biology, ≧99.0% (T),
Formic acid solution, 1.0 M in H₂O,
Gly-Gly-Gly, ≧99.0% (NT),
Gly-Gly, ≧99.5% (NT),
Glycine, ≧99.0% (NT),
Glycine, for luminescence, ≧99.0% (NT),
Glycine, for molecular biology, ≧99.0% (NT),
HEPES buffered saline, for molecular biology, 2× concentrate,
HEPES, ≧99.5% (T),
HEPES, for molecular biology, ≧99.5% (T),
Imidazole buffer Solution, 1 M in H₂O,
Imidazole, ≧99.5% (GC),
Imidazole, for luminescence, ≧99.5% (GC),
Imidazole, for molecular biology, ≧99.5% (GC),
Lipoprotein Refolding Buffer,
Lithium acetate dihydrate, ≧99.0% (NT),
Lithium citrate tribasic tetrahydrate, ≧99.5% (NT),
MES hydrate, ≧99.5% (T),
MES monohydrate, for luminescence, ≧99.5% (T),
MES solution, for molecular biology, 0.5 M in H₂O,
MOPS, ≧99.5% (T),
MOPS, for luminescence, ≧99.5% (T),
MOPS, for molecular biology, ≧99.5% (T),
Magnesium acetate solution, for molecular biology, ˜1 M in H₂O,
Magnesium acetate tetrahydrate, ≧99.0% (KT),
Magnesium citrate tribasic nonahydrate, ≧98.0% (calc. based on dry substance, KT),
Magnesium formate solution, 0.5 M in H₂O,
Magnesium phosphate dibasic trihydrate, ≧98.0% (KT),
Neutralization solution for the in-situ hybridization for in-situ hybridization, for molecular biology,
Oxalic acid dihydrate, ≧99.5% (RT),
PIPES, ≧99.5% (T),
PIPES, for molecular biology, ≧99.5% (T),
Phosphate buffered saline, solution (autoclaved),
Phosphate buffered saline, washing buffer for peroxidase conjugates in Western Blotting, 10× concentrate,
piperazine, anhydrous, ≧99.0% (T),
Potassium D-tartrate monobasic, ≧99.0% (T),
Potassium acetate solution, for molecular biology,
Potassium acetate solution, for molecular biology, 5 M in H₂O,
Potassium acetate solution, for molecular biology, ˜1 M in H₂O,
Potassium acetate, ≧99.0% (NT),
Potassium acetate, for luminescence, ≧99.0% (NT),
Potassium acetate, for molecular biology, ≧99.0% (NT),
Potassium bicarbonate, ≧99.5% (T),
Potassium carbonate, anhydrous, ≧99.0% (T),
Potassium chloride, ≧99.5% (AT),
Potassium citrate monobasic, ≧99.0% (dried material, NT),
Potassium citrate tribasic solution, 1 M in H₂O,
Potassium formate solution, 14 M in H₂O,
Potassium formate, ≧99.5% (NT),
Potassium oxalate monohydrate, ≧99.0% (RT),
Potassium phosphate dibasic, anhydrous, ≧99.0% (T),
Potassium phosphate dibasic, for luminescence, anhydrous, ≧99.0% (T),
Potassium phosphate dibasic, for molecular biology, anhydrous, ≧99.0% (T),
Potassium phosphate monobasic, anhydrous, ≧99.5% (T),
Potassium phosphate monobasic, for molecular biology, anhydrous, ≧99.5% (T),
Potassium phosphate tribasic monohydrate, 5% (T),
Potassium phthalate monobasic, ≧99.5% (T),
Potassium sodium tartrate solution, 1.5 M in H₂O,
Potassium sodium tartrate tetrahydrate, ≧99.5% (NT),
Potassium tetraborate tetrahydrate, ≧99.0% (T),
Potassium tetraoxalate dihydrate, ≧99.5% (RT),
Propionic acid solution, 1.0 M in H₂O,
STE buffer solution, for molecular biology, pH 7.8,
STET buffer solution, for molecular biology, pH 8.0,
Sodium 5,5-diethylbarbiturate, ≧99.5% (NT),
Sodium acetate solution, for molecular biology, ˜3 M in H₂O,
Sodium acetate trihydrate, ≧99.5% (NT),
Sodium acetate, anhydrous, ≧99.0% (NT),
Sodium acetate, for luminescence, anhydrous, ≧99.0% (NT),
Sodium acetate, for molecular biology, anhydrous, ≧99.0% (NT),
Sodium bicarbonate, ≧99.5% (T),
Sodium bitartrate monohydrate, ≧99.0% (T),
Sodium carbonate decahydrate, ≧99.5% (T),
Sodium carbonate, anhydrous, ≧99.5% (calc. on dry substance, T),
Sodium citrate monobasic, anhydrous, ≧99.5% (T),
Sodium citrate tribasic dihydrate, ≧99.0% (NT),
Sodium citrate tribasic dihydrate, for luminescence, ≧99.0% (NT),
Sodium citrate tribasic dihydrate, for molecular biology, ≧99.5% (NT),
Sodium formate solution, 8 M in H₂O,
Sodium oxalate, ≧99.5% (RT),
Sodium phosphate dibasic dihydrate, ≧99.0% (T),
Sodium phosphate dibasic dihydrate, for luminescence, ≧99.0% (T),
Sodium phosphate dibasic dihydrate, for molecular biology, ≧99.0% (T),
Sodium phosphate dibasic dodecahydrate, ≧99.0% (T),
Sodium phosphate dibasic solution, 0.5 M in H₂O,
Sodium phosphate dibasic, anhydrous, 99.5% (T),
Sodium phosphate dibasic, for molecular biology, ≧99.5% (T),
Sodium phosphate monobasic dihydrate, ≧99.0% (T),
Sodium phosphate monobasic dihydrate, for molecular biology, ≧99.0% (T),
Sodium phosphate monobasic monohydrate, for molecular biology, ≧99.5% (T),
Sodium phosphate monobasic solution, 5 M in H₂O,
Sodium pyrophosphate dibasic, ≧99.0% (T),
Sodium pyrophosphate tetrabasic decahydrate, ≧99.5% (T),
Sodium tartrate dibasic dihydrate, ≧99.0% (NT),
Sodium tartrate dibasic solution, 1.5 M in H₂O (colorless solution at 20° C.),
Sodium tetraborate decahydrate, ≧99.5% (T),
TAPS, ≧99.5% (T),
TES, ≧99.5% (calc. based on dry substance, T),
TM buffer solution, for molecular biology, pH 7.4,
TNT buffer solution, for molecular biology, pH 8.0,
TRIS Glycine buffer solution, 10× concentrate,
TRIS acetate-EDTA buffer solution, for molecular biology,
TRIS buffered saline, 10× concentrate,
TRIS glycine SDS buffer solution, for electrophoresis, 10× concentrate,
TRIS phosphate-EDTA buffer solution, for molecular biology, concentrate, 10× concentrate,
Tricine, ≧99.5% (NT),
Triethanolamine, ≧99.5% (GC),
Triethylamine, ≧99.5% (GC),
Triethylammonium acetate buffer, volatile buffer, ˜1.0 M in H₂O,
Triethylammonium phosphate solution, volatile buffer, ˜1.0 M in H₂O,
Trimethylammonium acetate solution, volatile buffer, ˜1.0 M in H₂O,
Trimethylammonium phosphate solution, volatile buffer, ˜1 M in H₂O,
Tris-EDTA buffer solution, for molecular biology, concentrate, 100× concentrate,
Tris-EDTA buffer solution, for molecular biology, pH 7.4,
Tris-EDTA buffer solution, for molecular biology, pH 8.0,
Trizma® acetate, ≧99.0% (NT),
Trizma® base, ≧99.8% (T),
Trizma® base, ≧99.8% (T),
Trizma® base, for luminescence, ≧99.8% (T),
Trizma® base, for molecular biology, ≧99.8% (T),
Trizma® carbonate, ≧98.5% (T),
Trizma® hydrochloride buffer solution, for molecular biology, pH 7.2,
Trizma® hydrochloride buffer solution, for molecular biology, pH 7.4,
Trizma® hydrochloride buffer solution, for molecular biology, pH 7.6,
Trizma® hydrochloride buffer solution, for molecular biology, pH 8.0,
Trizma® hydrochloride, ≧999.0% (AT),
Trizma® hydrochloride, for luminescence, ≧99.0% (AT),
Trizma® hydrochloride, for molecular biology, ≧99.0% (AT), and
Trizma® maleate, ≧99.5% (NT); or
(d) any combination thereof.

29. The method of claim 1, wherein the water is present in Phosphate Buffered Saline (PBS).

30. The method of claim 1, wherein the nanoemulsion is topically applied:
(a) in a single administration;
(b) for at least once a week, at least twice a week, at least once a day, at least twice a day, multiple times daily, multiple times weekly, biweekly, at least once a month, or any combination thereof;
(c) for a period of time selected from the group consisting of about one week, about two weeks, about three weeks, about one month, about two months, about three months, about four months, about five months, about six months, about seven months, about eight months, about nine months, about ten months, about eleven months, about one year, about 1.5 years, about 2 years, about 2.5 years, about 3 years, about 3.5 years, about 4 years, about 4.5 years, and about 5 years;
(d) followed by washing the application area to remove any residual nanoemulsion; or
(e) any combination thereof.

31. The method of claim 1, wherein the nanoemulsion droplets enter the pilosebaeous gland (unit), hair follicle, epidermis, dermis, or a combination thereof.

32. The method of claim 1, wherein the nanoemulsion is a controlled release formulation, sustained release formulation, immediate release formulation, or any combination thereof.

33. The method of claim 1, wherein the nanoemulsion further comprises at least one anti-acne agent.

34. The method of claim 33, wherein the anti-acne agent is selected from the group consisting of benzoyl peroxide, salicylic acid and retinoid.
